(19) United States
(12) Patent Application Publication
(10) Pub. No.: US 2023/0247904 A1
(43) Pub. Date: Aug. 3, 2023

(54) NOVEL COMPOUND AND ORGANIC LIGHT EMITTING DEVICE COMPRISING THE SAME

(71) Applicant: LG CHEM, LTD., Seoul (KR)

(72) Inventors: Min Woo JUNG, Daejeon (KR); Dong Hoon LEE, Daejeon (KR); Sang Duk SUH, Daejeon (KR); Jungha LEE, Daejeon (KR); Su Jin HAN, Daejeon (KR); Seulchan PARK, Daejeon (KR); Sunghyun HWANG, Daejeon (KR)

(21) Appl. No.: 17/926,724

(22) PCT Filed: Aug. 4, 2021
(86) PCT No.: PCT/KR2021/010245

§ 371 Date: Nov. 21, 2022

(30) Foreign Application Priority Data
Aug. 4, 2020 (KR) 10-2020-0097601
Aug. 3, 2021 (KR) 10-2021-0101872

(51) Int. Cl. H10K 85/60; C07D 405/14; C09K 11/06; C07D 409/14

(52) U.S. Cl. CPC H10K 85/6574; C07D 405/14; C09K 11/06; H10K 85/654; H10K 85/6572; C07D 409/14; H10K 50/11

(57) ABSTRACT

Provided is a compound of Chemical Formula 1:

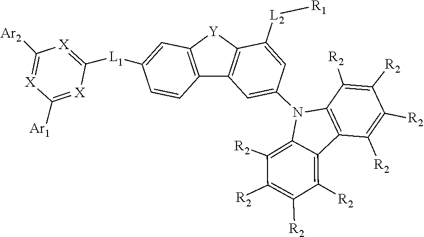

wherein: each X is independently N or CH, with two or more of the X being N; Y is O or S; and the other substituents are as described in the specification, where at least one of Ar₁, Ar₂ and R₁ is substituted with at least one deuterium, or at least one of R₂ is deuterium; and an organic light-emitting device including the same. When the compound is used as an organic light-emitting layer material, it exhibits excellent characteristics in terms of efficiency and lifetime. This is considered to be because the core dibenzofuran or dibenzothiophene group is substituted with the triazine and carbazole groups, and when at least one of Ar₁, Ar₂ and R₁ is substituted with at least one deuterium, or at least one of R₂ is deuterium, the compound exhibits increased electronic stability.

### CROSS-REFERENCE TO RELATED APPLICATION(S)

This application is a National Stage Application of International Application No. PCT/KR2021/010245 filed on Aug. 4, 2021, which claims the benefit of Korean Patent Application No. 10-2020-0097601 filed on Aug. 4, 2020 and Korean Patent Application No. 10-2021-0101872 filed on Aug. 3, 2021 in the Korean Intellectual Property Office, the disclosures of which are incorporated herein by reference in their entirety.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of Chemical Formula 1:

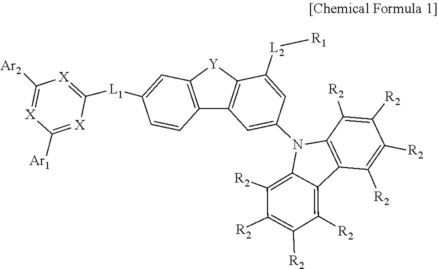

wherein, in Chemical Formula 1:

each X is independently N or CH, with two or more of the X being N;

Y is O or S;

L₁ is a direct bond or a substituted or unsubstituted C₆₋₆₀ arylene;

L₂ is a direct bond or a substituted or unsubstituted C₆₋₆₀ arylene;

Ar₁ and Ar₂ are each independently a substituted or unsubstituted C₆₋₆₀ aryl, or a substituted or unsubstituted C₆₋₆₀ heteroaryl containing any one or more heteroatoms selected from the group consisting of N, O and S;

R₁ is a substituted or unsubstituted C₆₋₆₀ aryl, or a substituted or unsubstituted C₆₋₆₀ heteroaryl containing any one or more heteroatoms selected from the group consisting of N, O and S; and

each R₂ is independently hydrogen or deuterium;

with the proviso that at least one of Ar₁, Ar₂ and R₁ is substituted with at least one deuterium, or at least one of R₂ is deuterium.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device.

In particular, the above-mentioned compound of Chemical Formula 1 can be used as a hole injection material, hole transport material, hole injection and transport material, light emitting material, electron transport material, or electron injection material.

### BRIEF DESCRIPTION OF THE DRAWINGS

FIG. 1 shows an example of an organic light emitting device comprising a substrate 1, an anode 2, a hole transport layer 3, a light emitting layer 4, an electron injection and transport layer 5, and a cathode 6.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6.

### DETAILED DESCRIPTION

Hereinafter, embodiments of the present disclosure will be described in more detail to facilitate understanding of the invention.

### DEFINITION OF TERMS

As used herein, the notation [symbol] and [symbol] mean a bond linked to another substituent group.

As used herein, the term “substituted or unsubstituted” means being unsubstituted or substituted with one or more substituents selected from the group consisting of deuterium, a halogen group, a cyano group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can also be interpreted as a substituent in which two phenyl groups are linked.

In the present disclosure, the carbon number of a carbonyl group is not particularly limited, but is preferably 1 to 40. Specifically, the carbonyl group can be a compound having the following structural formulas, but is not limited thereto:

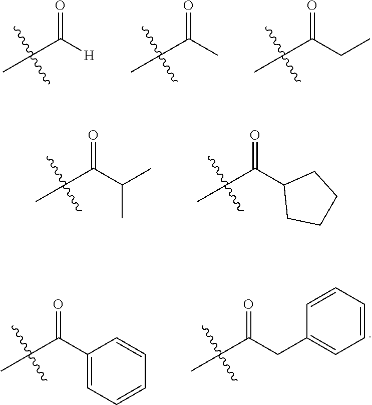

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a compound having the following structural formulas, but is not limited thereto:

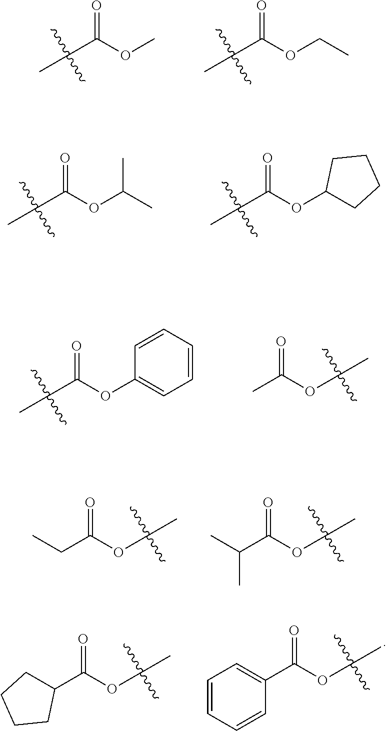

In the present disclosure, the carbon number of an imide group is not particularly limited, but is preferably 1 to 25. Specifically, the imide group can be a compound having the following structural formulas, but is not limited thereto:

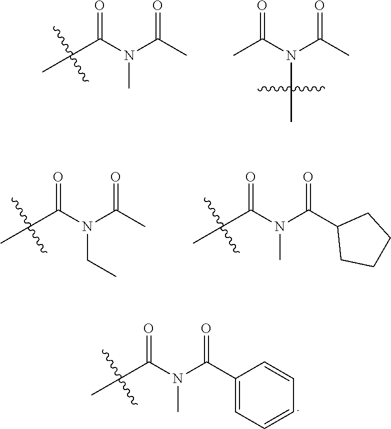

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group having aromaticity. According to one embodiment, the carbon number of the aryl group is 6 to 30. According to one embodiment, the carbon number of the aryl group is 6 to 20. The aryl group can be a phenyl group, a biphenylyl group, a terphenylyl group or the like as the monocyclic aryl group, but is not limited thereto. The polycyclic aryl group includes a naphthyl group, an anthracenyl group, a phenanthrenyl group, a triphenylenyl group, a pyrenyl group, a perylenyl group, a chrysenyl group, or the like, but is not limited thereto.

In the present disclosure, a heteroaryl is a heteroaryl containing at least one of O, N, Si and S as a heteroatom, and the carbon number thereof is not particularly limited, but is preferably 2 to 60. Examples of the heteroaryl include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the aryl silyl group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heteroaryl. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heteroaryl can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the above-mentioned description of the heteroaryl can be applied except that the heterocycle is not a monovalent group but formed by combining two substituent groups.

(Compound)

The present disclosure provides a compound of Chemical Formula 1.

Hereinafter, the Chemical Formula 1 and the compound of Chemical Formula 1 are described in detail as follows.

Each X is independently N or CH, with two or more of the X being N.

Specifically, all X can be N.

Y can be O or S, for example O.

L₁ can be a direct bond, a substituted or unsubstituted C₆₋₆₀ arylene. For example, L₁ can be a direct bond, phenylene, biphenylene, or naphthylene.

L₂ can be a direct bond, a substituted or unsubstituted C₆₋₆₀ arylene. For example, L₂ can be a direct bond or phenylene.

Ar₁ and Ar₂ can be each independently a substituted or unsubstituted C₆₋₆₀ aryl, or a substituted or unsubstituted C₆₋₆₀ heteroaryl containing any one or more heteroatoms selected from the group consisting of N, O and S.

Specifically, Ar₁ and Ar₂ are each independently phenyl, biphenylyl, naphthyl, naphthyl phenyl, phenyl naphthyl, phenanthrenyl, carbazol-9-yl, 9-phenyl-9H-carbazolyl, dibenzofuranyl, or dibenzothiophenyl, wherein the Ar₁ and Ar₂ can be unsubstituted or substituted with at least one deuterium.

For example, Ar₁ and Ar₂ can be each independently unsubstituted biphenylyl, naphthyl, naphthyl phenyl, phenyl naphthyl, phenanthrenyl, carbazol-9-yl, 9-phenyl-9H-carbazolyl, dibenzofuranyl, dibenzothiophenyl, or phenyl unsubstituted or substituted with 5 deuteriums.

R₁ can be a substituted or unsubstituted C₆₋₆₀ aryl, or a substituted or unsubstituted C₆₋₆₀ heteroaryl containing any one or more heteroatoms selected from the group consisting of N, O and S.

Specifically, R₁ can be phenyl, biphenylyl, terphenylyl, naphthyl, phenanthrenyl, dibenzofuranyl, or dibenzothiophenyl, wherein the R₁ is unsubstituted or substituted with at least one deuterium.

For example, R₁ can be biphenylyl, terphenylyl, naphthyl, phenanthrenyl, dibenzofuranyl, dibenzothiophenyl, or phenyl unsubstituted or substituted with 5 deuteriums.

Each R₂ can be independently hydrogen or deuterium.

However, Chemical Formula 1 satisfies the above definition, and at the same time, at least one of Ar₁, Ar₂ and R₁ is substituted with at least one deuterium, or at least one of R₂ is deuterium.

As a more specific example, the compound of Chemical Formula 1 can be any one compound selected from the group consisting of the following compounds:

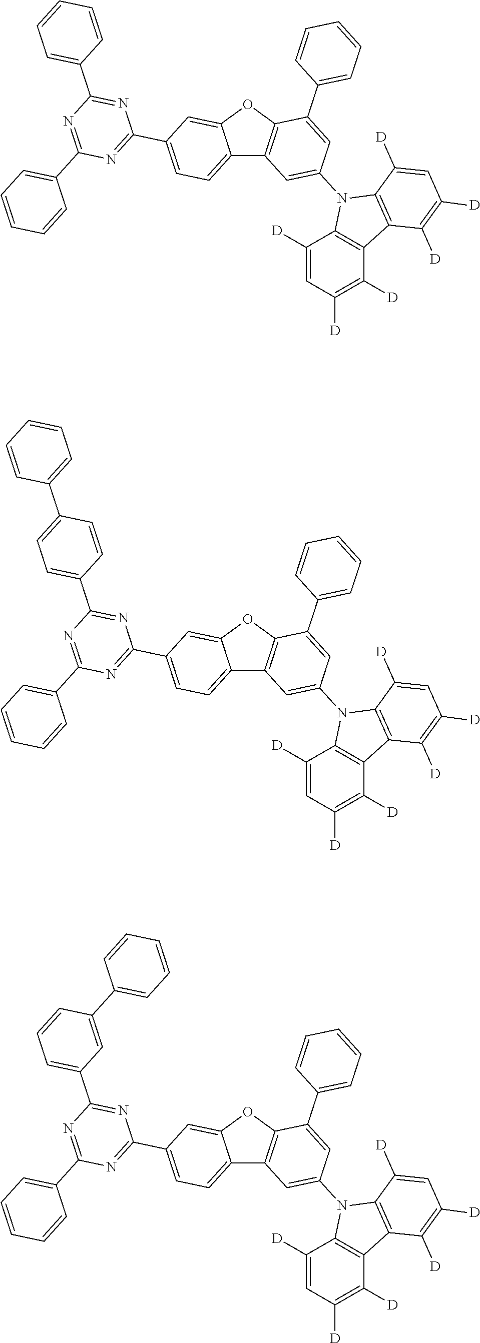 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6ccccc6)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc(-c6ccccc6)c5)n4)ccc3c2c1]

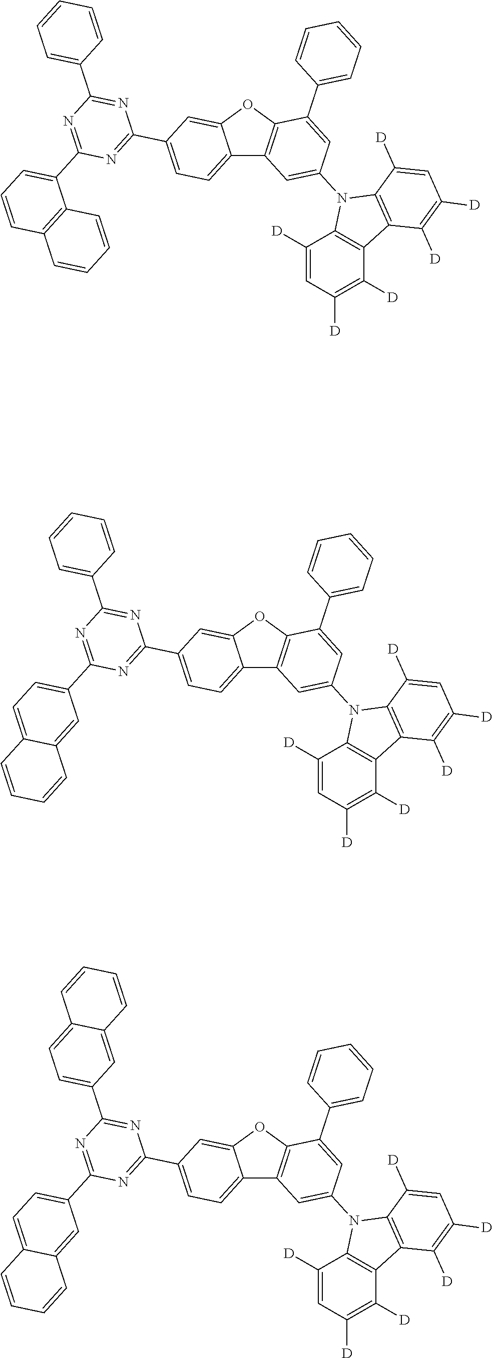 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6ccccc6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccc6ccccc6c5)nc(-c5ccc6ccccc6c5)n4)ccc3c2c1]

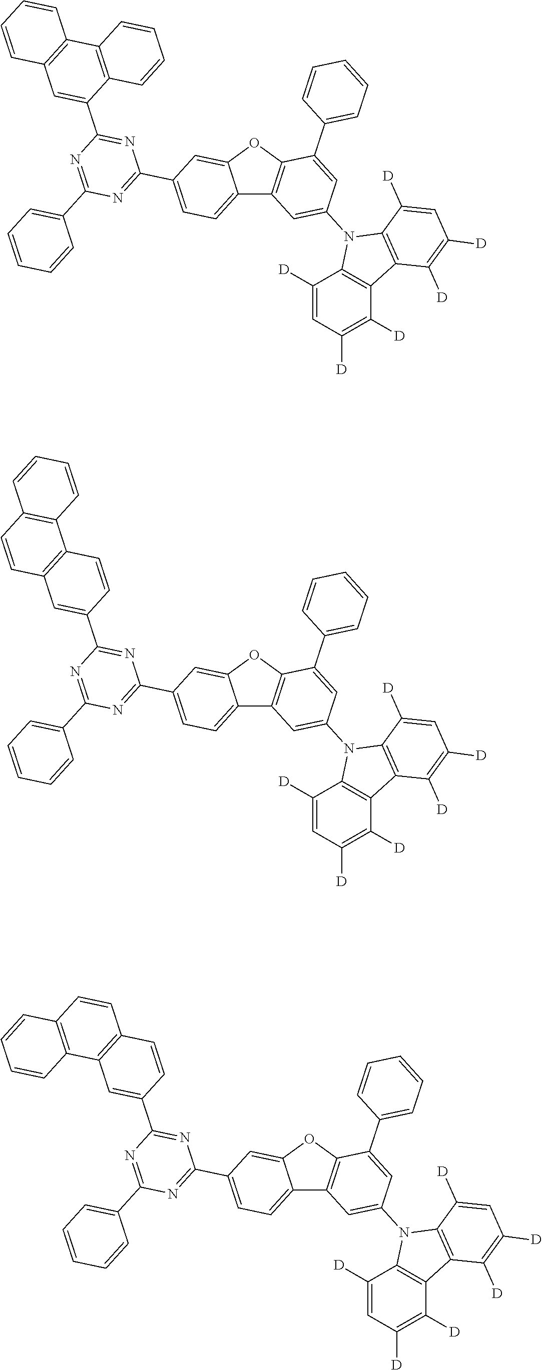 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5cc6ccccc6c6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(ccc7ccccc76)c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6ccc7ccccc7c6c5)n4)ccc3c2c1]

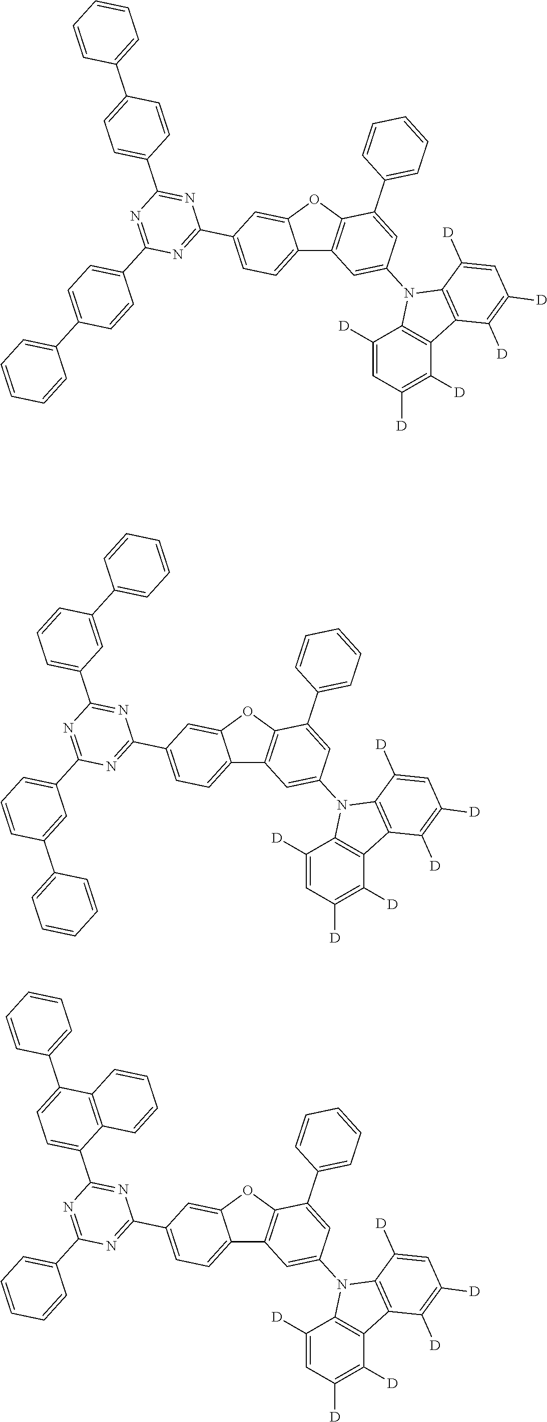 [SMILES: [2H]c1cc([2H])c2c(c1[2H])c1c([2H])c([2H])cc([2H])c1n2-c1cc(-c2ccccc2)c2oc3cc(-c4nc(-c5ccc(-c6ccccc6)cc5)nc(-c5ccc(-c6ccccc6)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5cccc(-c6ccccc6)c5)nc(-c5cccc(-c6ccccc6)c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6ccccc6)c6ccccc56)n4)ccc3c2c1]

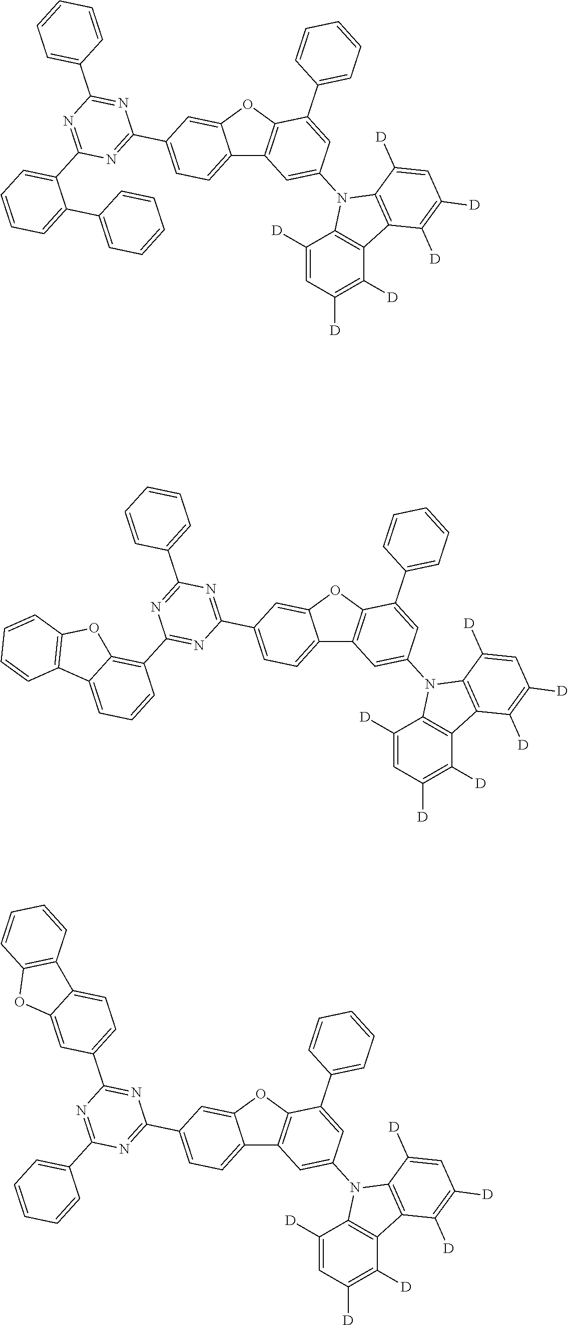 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccccc5-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5oc5ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)oc5ccccc56)n4)ccc3c2c1]

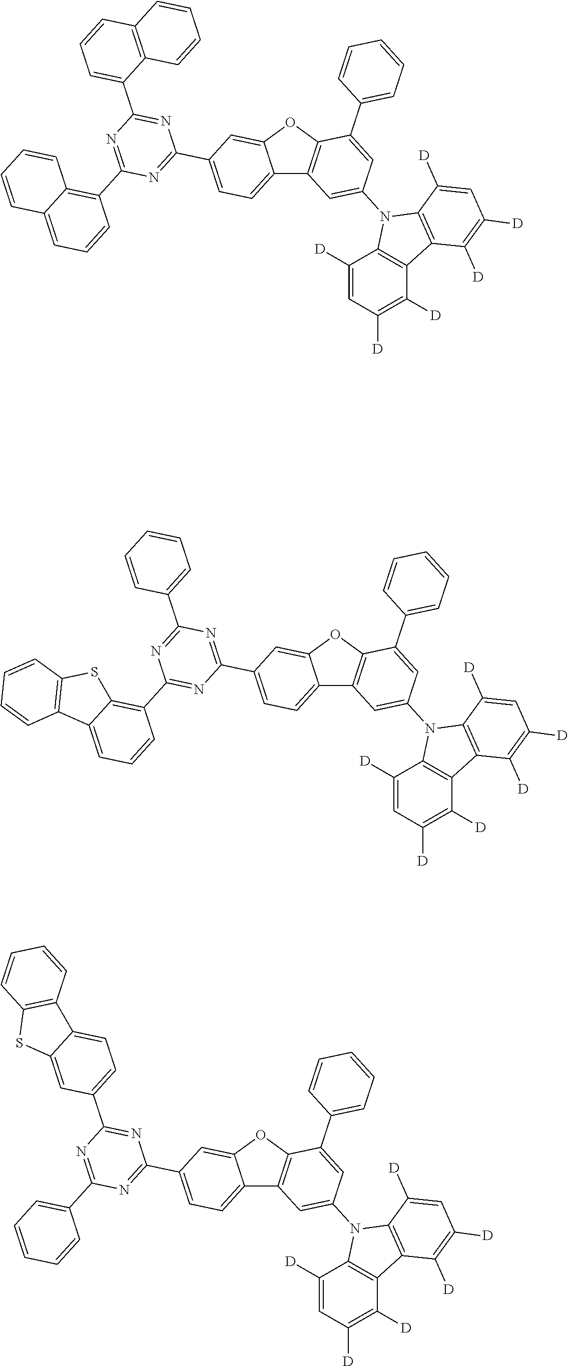 [SMILES: [2H]c1cc([2H])c2c(c1[2H])c1c([2H])c([2H])cc([2H])c1n2-c1cc(-c2ccccc2)c2oc3cc(-c4nc(-c5cccc6ccccc56)nc(-c5cccc6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5sc5ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)sc5ccccc56)n4)ccc3c2c1]

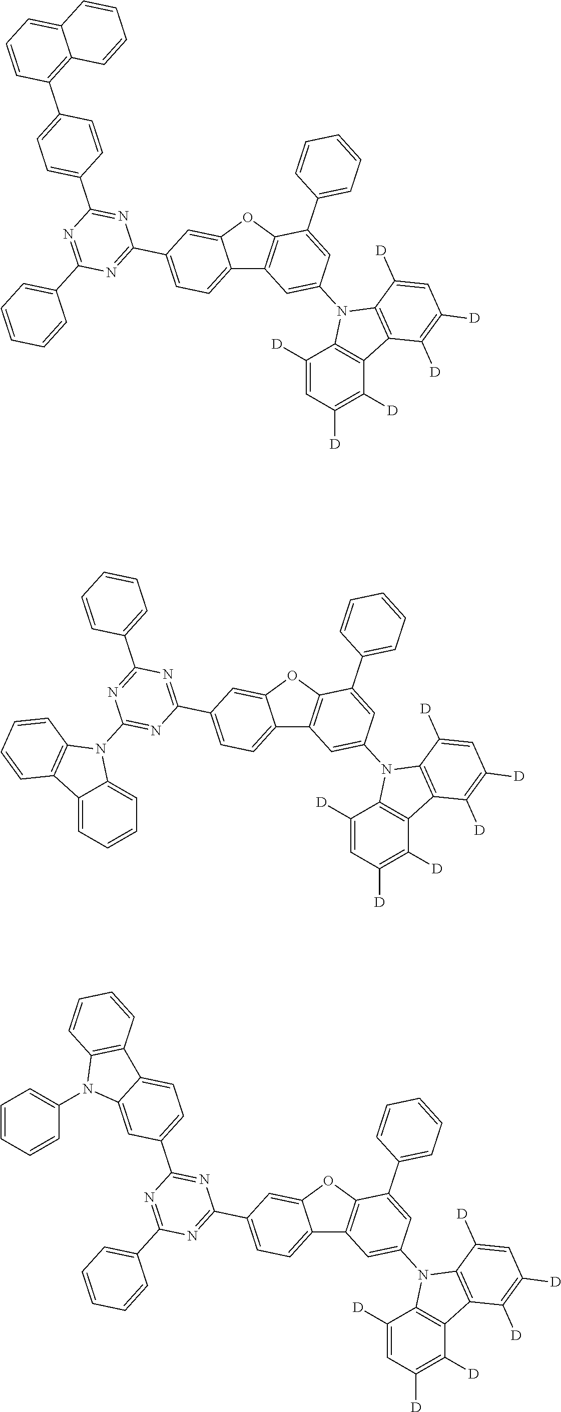 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6cccc7ccccc67)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-n5c6ccccc6c6ccccc65)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c7ccccc7n(-c7ccccc7)c6c5)n4)ccc3c2c1]

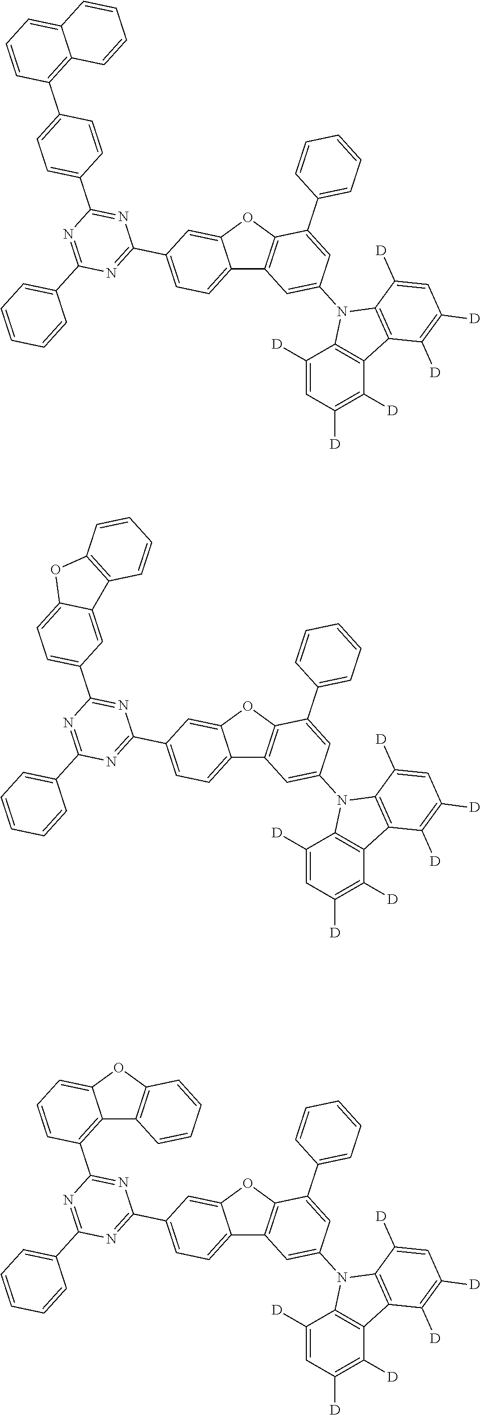 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6cccc7ccccc67)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6oc7ccccc7c6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6oc7ccccc7c56)n4)ccc3c2c1]

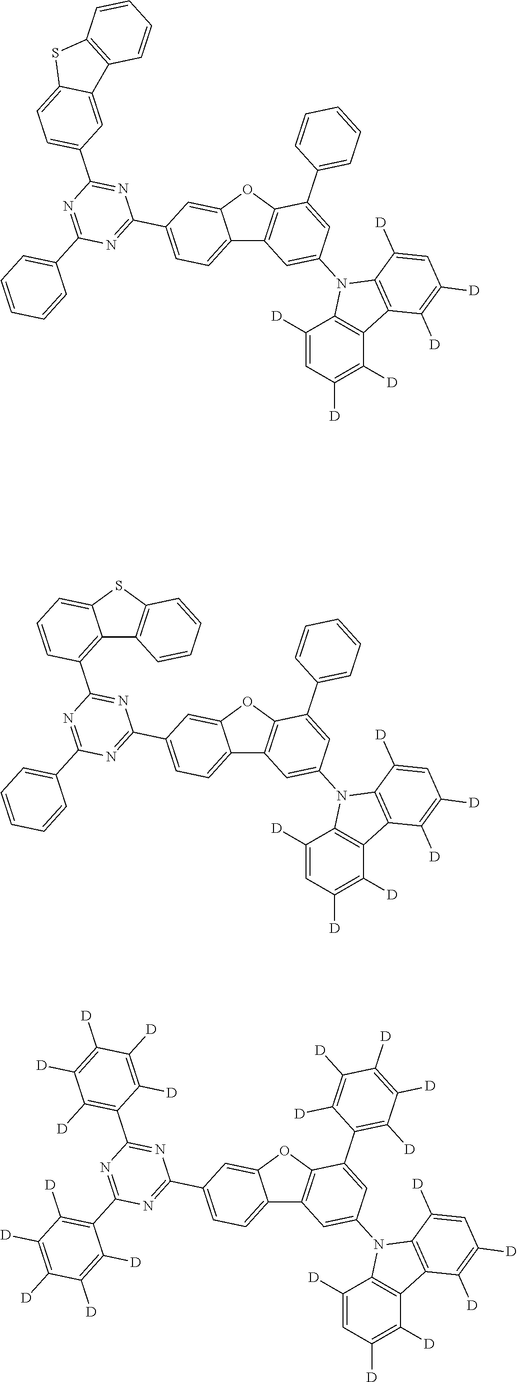 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6sc7ccccc7c6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6sc7ccccc7c56)n4)ccc3c2c1 ; [2H]c1cc([2H])c2c(c1[2H])c1c([2H])c([2H])cc([2H])c1n2-c1cc(-c2c([2H])c([2H])c([2H])c([2H])c2[2H])c2oc3cc(-c4nc(-c5c([2H])c([2H])c([2H])c([2H])c5[2H])nc(-c5c([2H])c([2H])c([2H])c([2H])c5[2H])n4)ccc3c2c1]

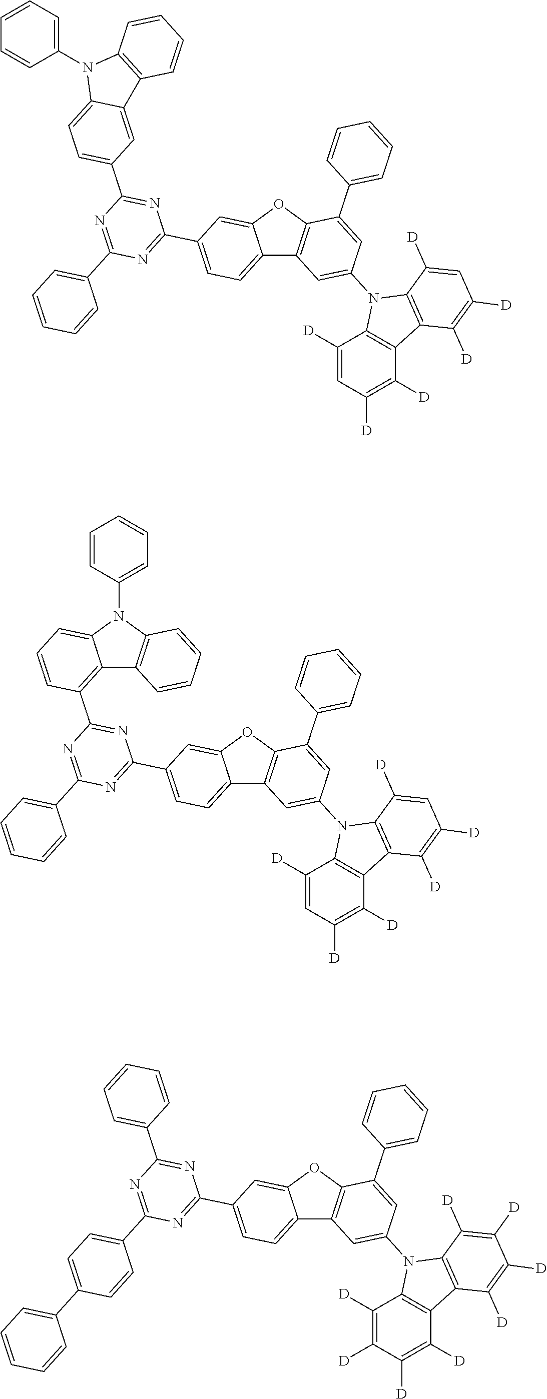 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)c5ccccc5n6-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5c5ccccc5n6-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccc(-c6ccccc6)cc5)n4)ccc3c2c1]

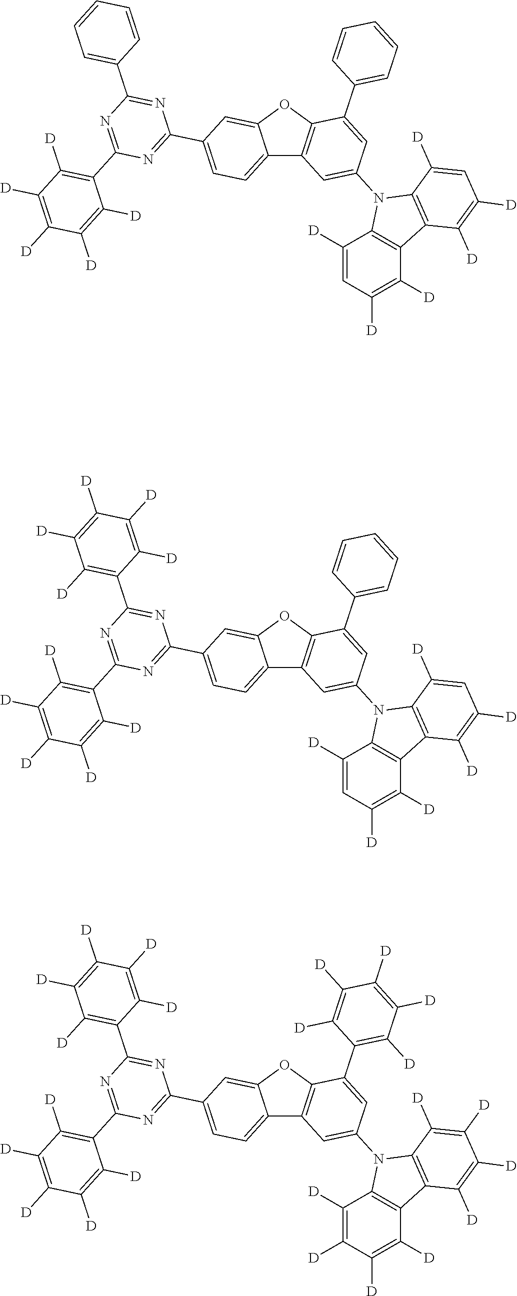

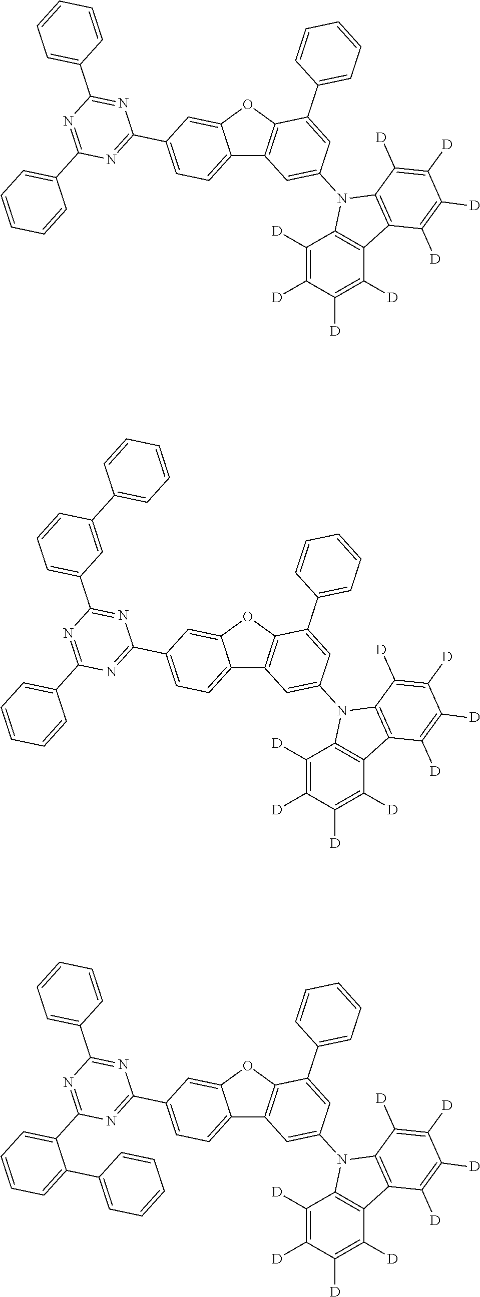 [SMILES: [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5cccc(-c6ccccc6)c5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5-c5ccccc5)n4)ccc3c2c1]

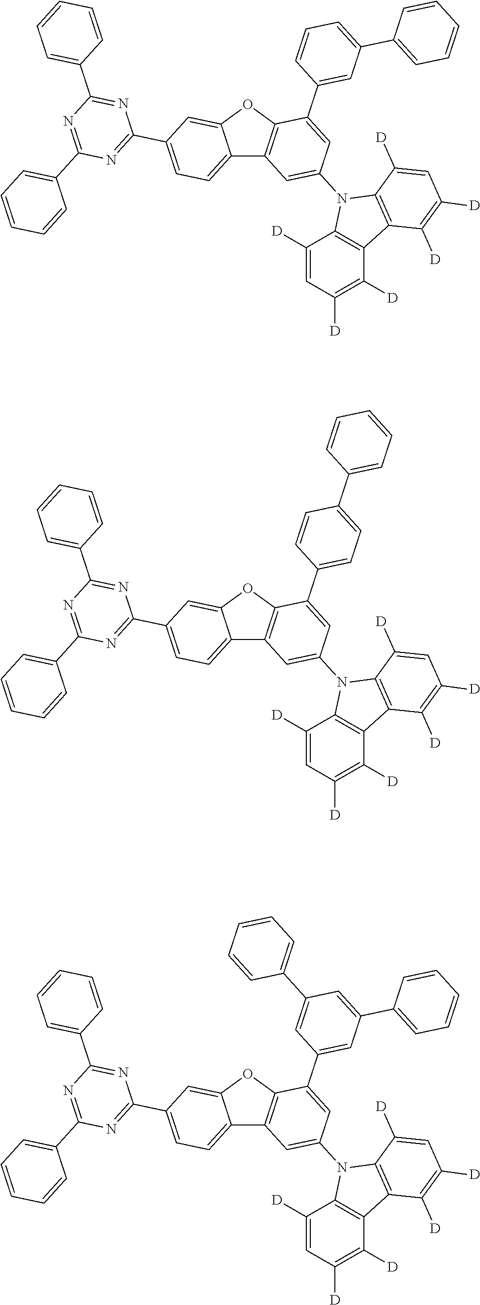 [SMILES: [2H]c1cc([2H])c2c(c1[2H])c1c([2H])c([2H])cc([2H])c1n2-c1cc(-c2cccc(-c3ccccc3)c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc(-c3ccccc3)cc2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cc(-c3ccccc3)cc(-c3ccccc3)c2)c2oc3cc(-c4nc(-c5ccccc5)nc(-c5ccccc5)n4)ccc3c2c1]

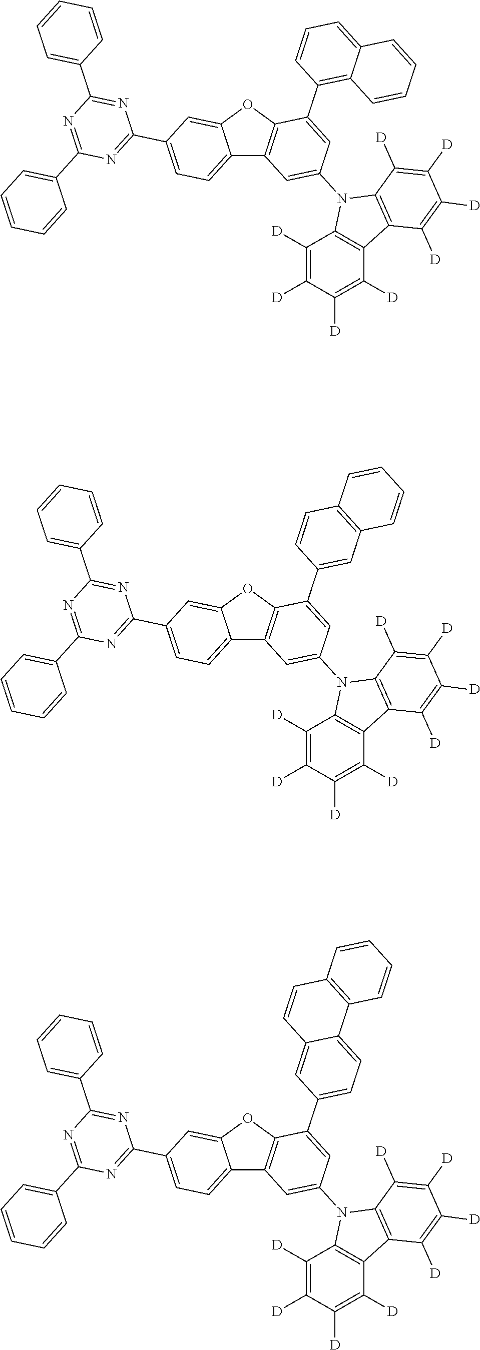 [SMILES: [2H]c1c([2H])c([2H])c2c(c1[2H])c1c([2H])c([2H])c([2H])c([2H])c1n2-c1cc(-c2cccc3ccccc23)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccc3ccccc3c2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccc3c(ccc4ccccc43)c2)c2oc3cc(-c4nc(-c5ccccc5)nc(-c5ccccc5)n4)ccc3c2c1]

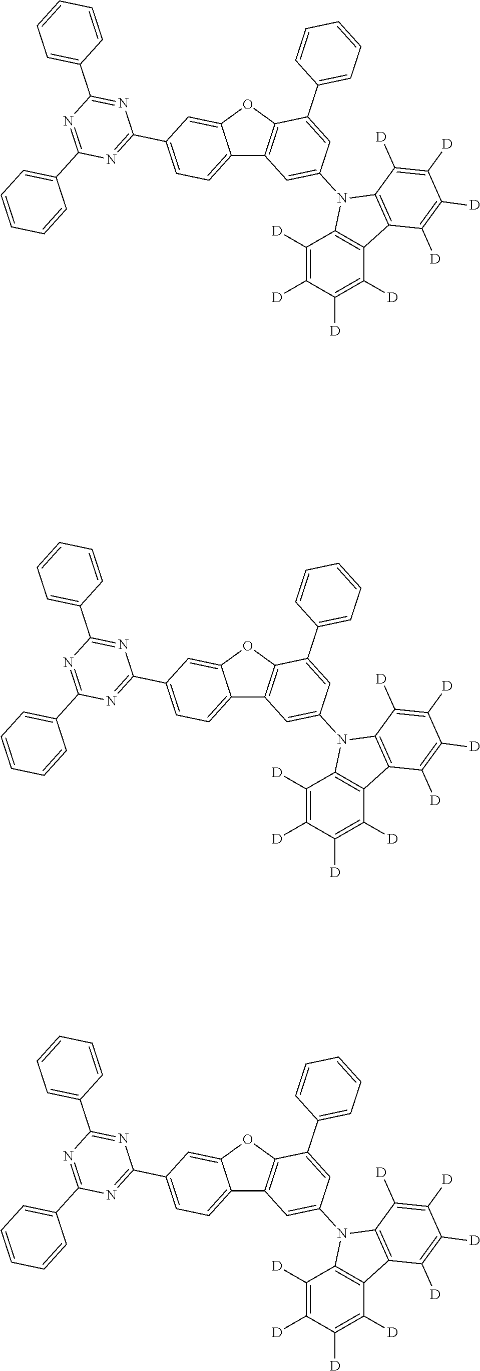 [SMILES: [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1]

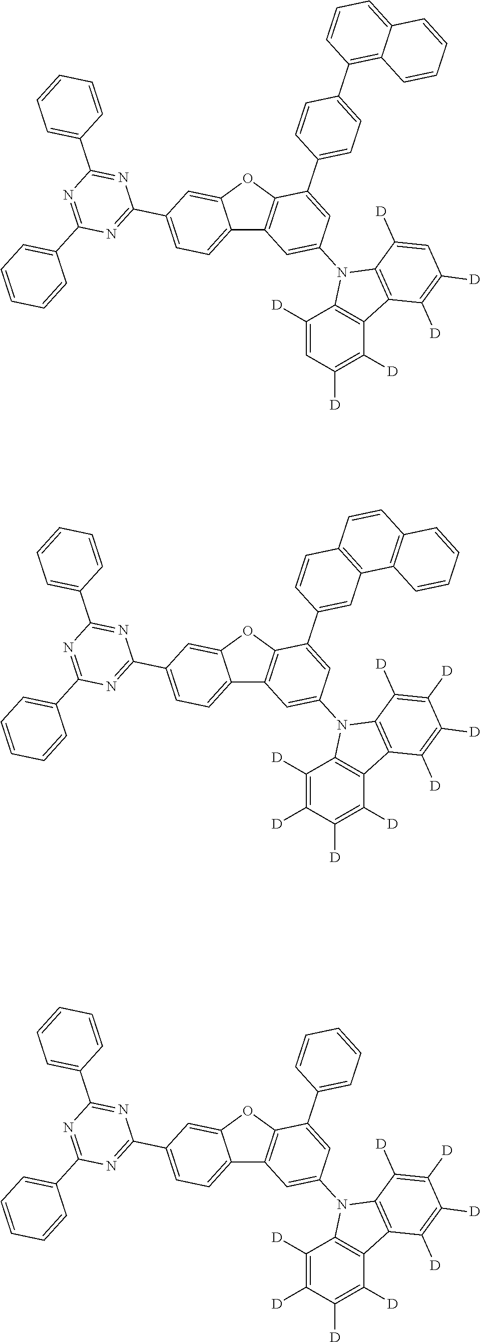 [SMILES: [2H]c1cc([2H])c2c(c1[2H])c1c([2H])c([2H])cc([2H])c1n2-c1cc(-c2ccc(-c3cccc4ccccc34)cc2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccc3ccc4ccccc4c3c2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1]

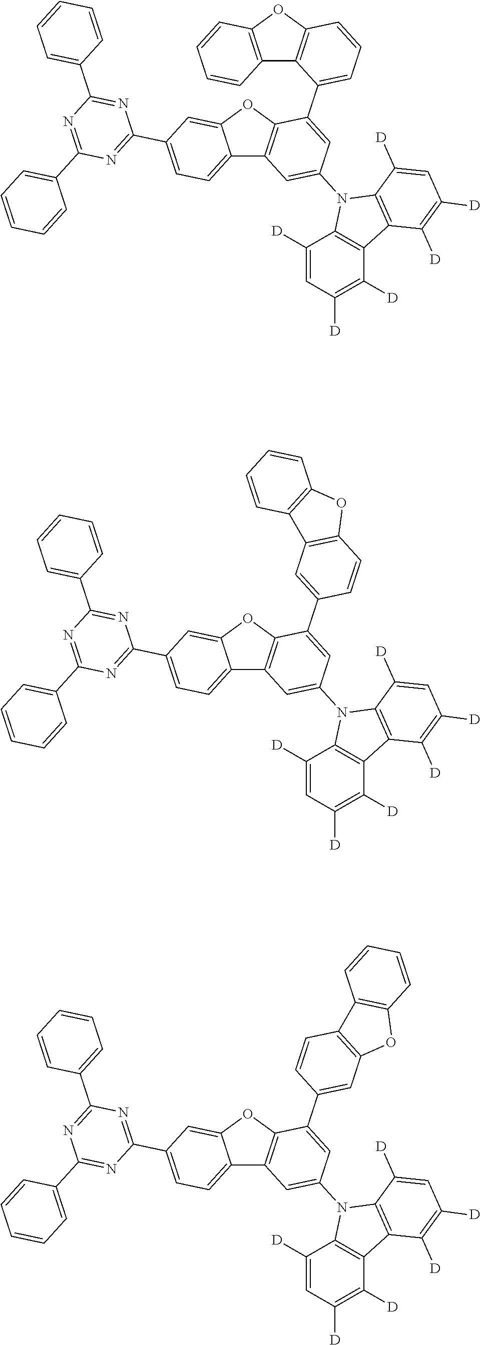 [SMILES: [2H]c1cc([2H])c2c(c1[2H])c1c([2H])c([2H])cc([2H])c1n2-c1cc(-c2cccc3oc4ccccc4c23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3oc4ccccc4c3c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3c(c2)oc2ccccc23)c2oc3cc(-c4nc(-c5ccccc5)nc(-c5ccccc5)n4)ccc3c2c1]

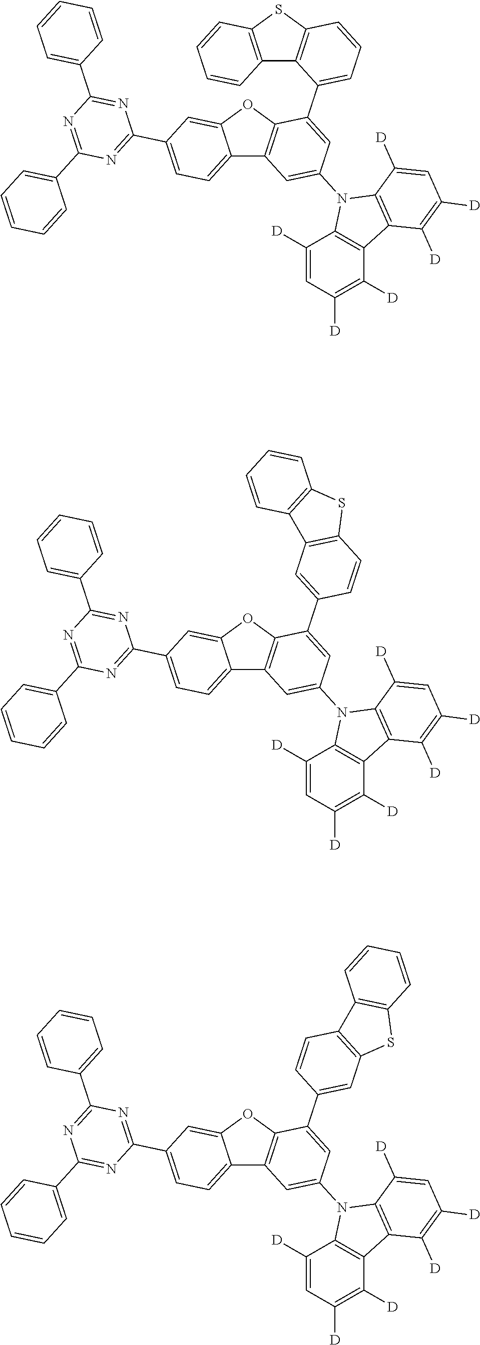 [SMILES: [2H]c1cc([2H])c2c(c1[2H])c1c([2H])c([2H])cc([2H])c1n2-c1cc(-c2cccc3sc4ccccc4c23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3sc4ccccc4c3c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3c(c2)sc2ccccc23)c2oc3cc(-c4nc(-c5ccccc5)nc(-c5ccccc5)n4)ccc3c2c1]

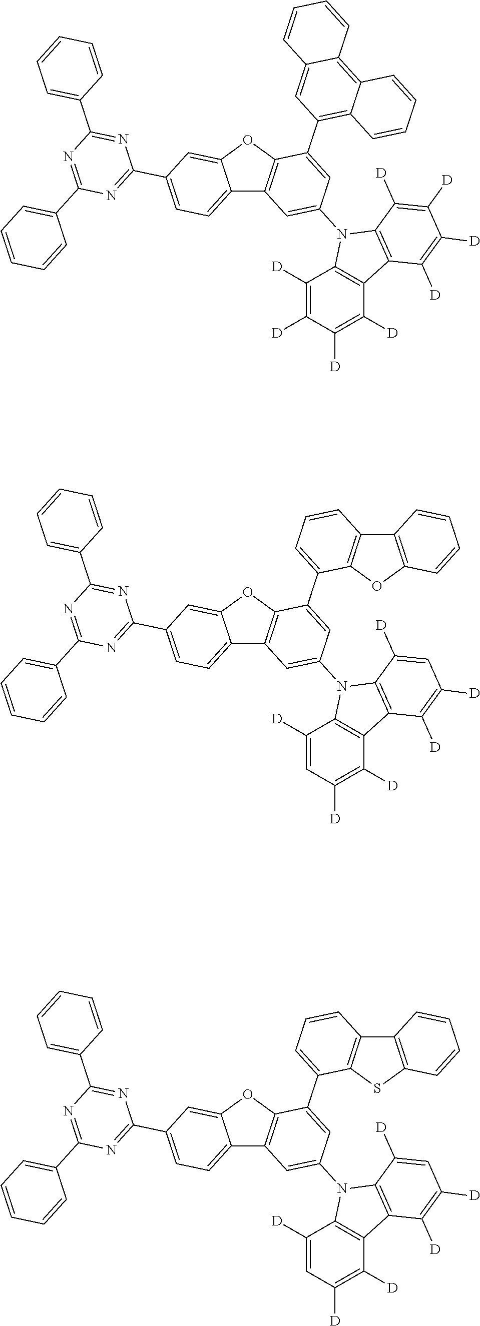 [SMILES: [2H]c1c([2H])c([2H])c2c(c1[2H])c1c([2H])c([2H])c([2H])c([2H])c1n2-c1cc(-c2cc3ccccc3c3ccccc23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cccc3c2oc2ccccc23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cccc3c2sc2ccccc23)c2oc3cc(-c4nc(-c5ccccc5)nc(-c5ccccc5)n4)ccc3c2c1]

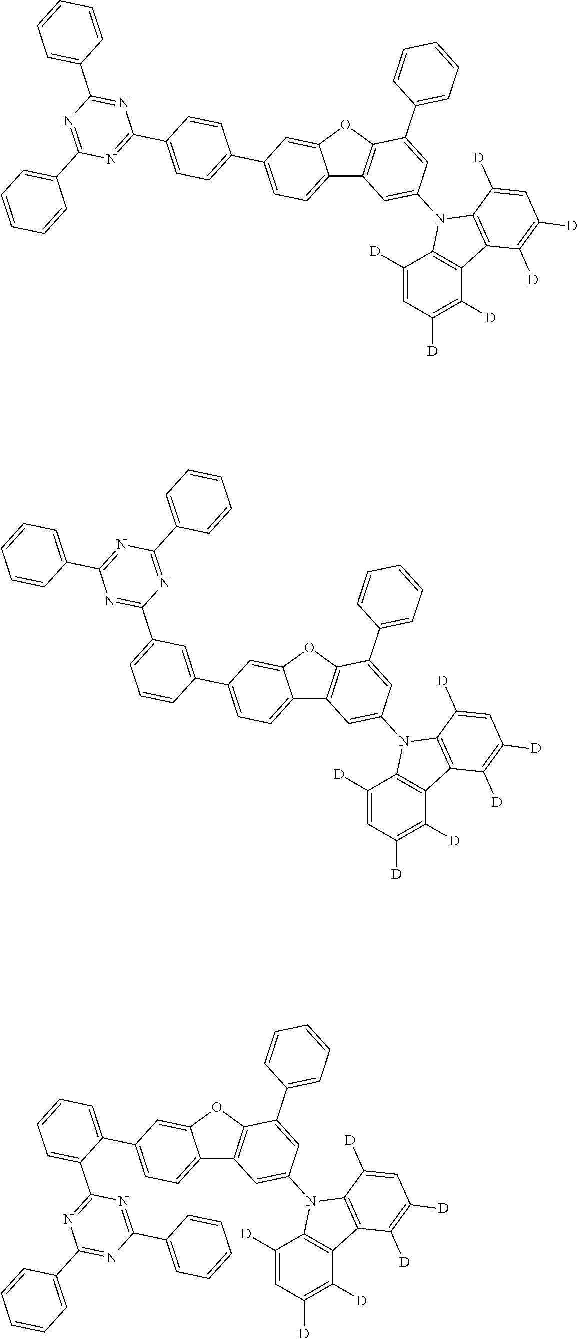 [SMILES: [2H]c1cc([2H])c2c(c1[2H])c1c([2H])c([2H])cc([2H])c1n2-c1cc(-c2ccccc2)c2oc3cc(-c4ccc(-c5nc(-c6ccccc6)nc(-c6ccccc6)n5)cc4)ccc3c2c1 ; [2H]c1cc([2H])c2c(c1[2H])c1c([2H])c([2H])cc([2H])c1n2-c1cc(-c2ccccc2)c2oc3cc(-c4cccc(-c5nc(-c6ccccc6)nc(-c6ccccc6)n5)c4)ccc3c2c1 ; [2H]c1cc([2H])c2c(c1[2H])c1c([2H])c([2H])cc([2H])c1n2-c1cc(-c2ccccc2)c2oc3cc(-c4ccccc4-c4nc(-c5ccccc5)nc(-c5ccccc5)n4)ccc3c2c1]

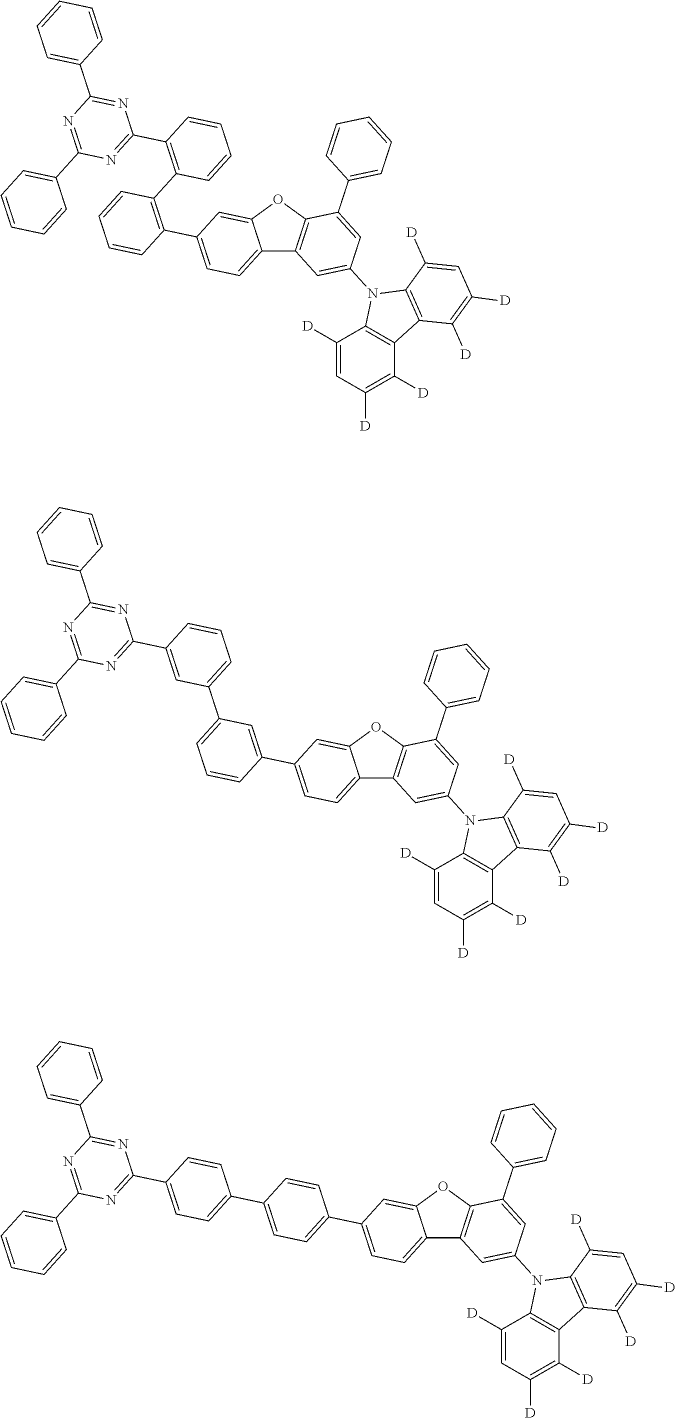 [SMILES: [2H]c1cc([2H])c2c(c1[2H])c1c([2H])c([2H])cc([2H])c1n2-c1cc(-c2ccccc2)c2oc3cc(-c4ccccc4-c4ccccc4-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4cccc(-c5cccc(-c6nc(-c7ccccc7)nc(-c7ccccc7)n6)c5)c4)ccc3c2c1 ; [2H]c1cc([2H])c2c(c1[2H])c1c([2H])c([2H])cc([2H])c1n2-c1cc(-c2ccccc2)c2oc3cc(-c4ccc(-c5ccc(-c6nc(-c7ccccc7)nc(-c7ccccc7)n6)cc5)cc4)ccc3c2c1]

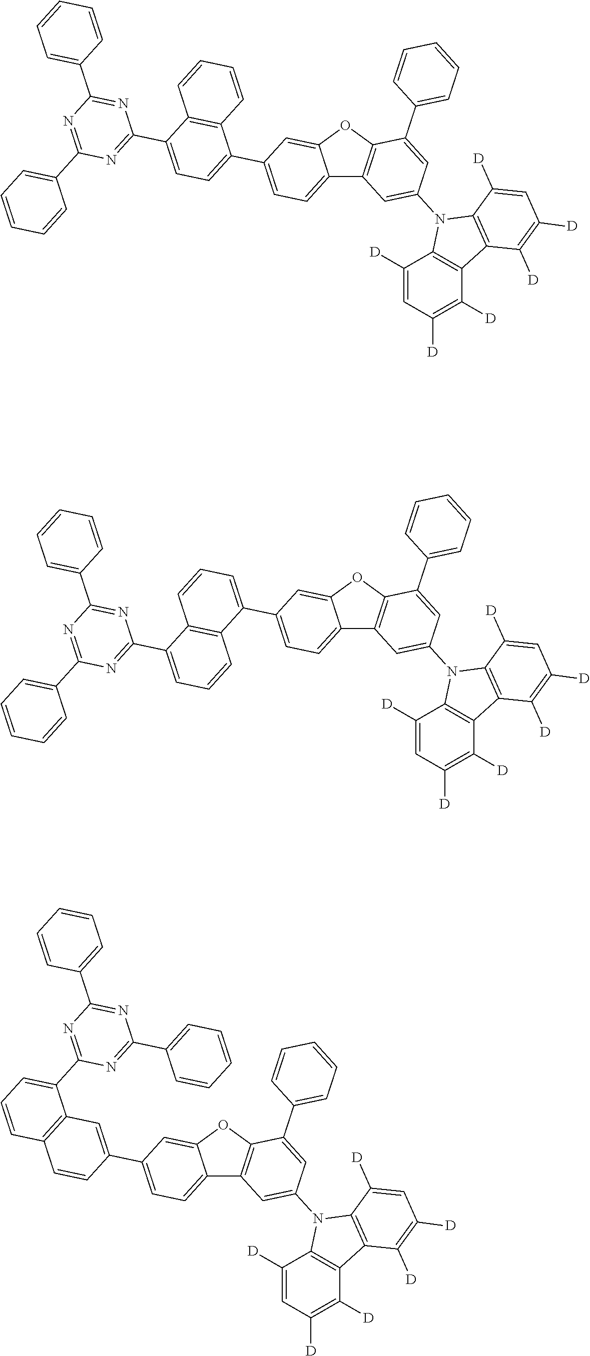 [SMILES: [2H]c1cc([2H])c2c(c1[2H])c1c([2H])c([2H])cc([2H])c1n2-c1cc(-c2ccccc2)c2oc3cc(-c4ccc(-c5nc(-c6ccccc6)nc(-c6ccccc6)n5)c5ccccc45)ccc3c2c1 ; [2H]c1cc([2H])c2c(c1[2H])c1c([2H])c([2H])cc([2H])c1n2-c1cc(-c2ccccc2)c2oc3cc(-c4cccc5c(-c6nc(-c7ccccc7)nc(-c7ccccc7)n6)cccc45)ccc3c2c1 ; [2H]c1cc([2H])c2c(c1[2H])c1c([2H])c([2H])cc([2H])c1n2-c1cc(-c2ccccc2)c2oc3cc(-c4ccc5cccc(-c6nc(-c7ccccc7)nc(-c7ccccc7)n6)c5c4)ccc3c2c1]

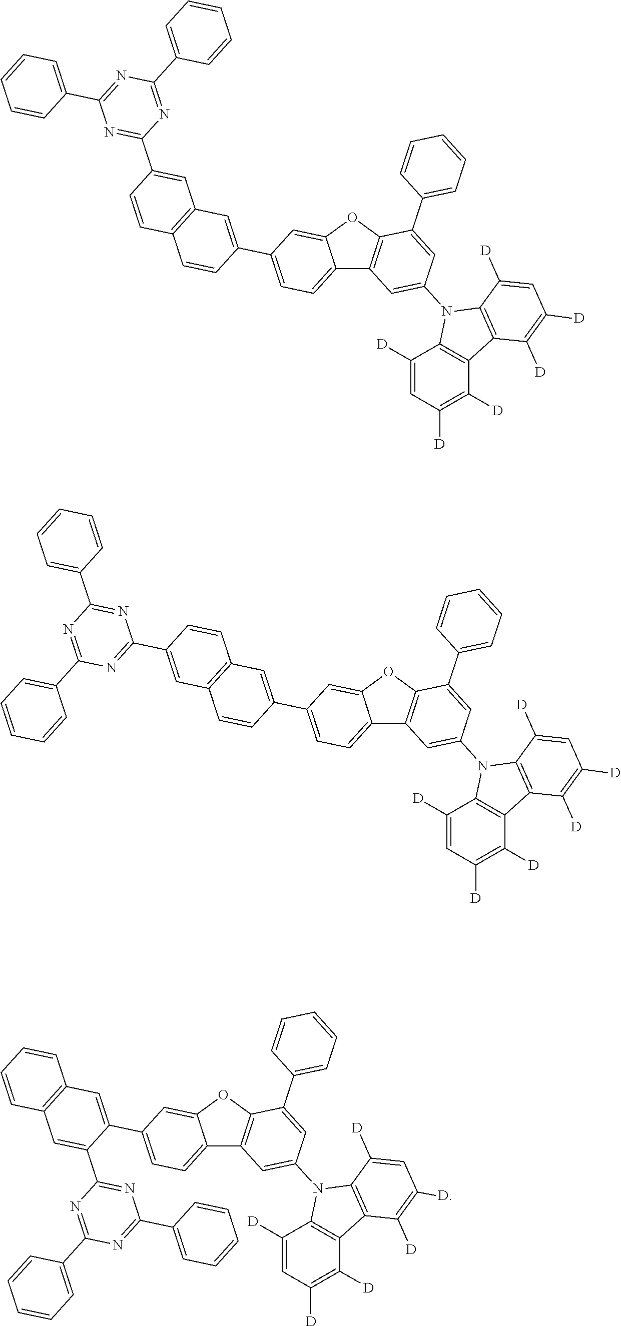

In addition, the present disclosure provides a method for preparing the compound of Chemical Formula 1 as shown in the following Reaction Scheme 1.

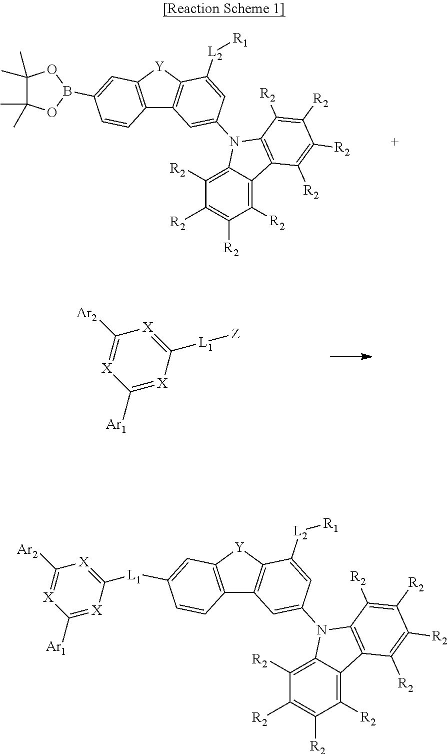

In Reaction Scheme 1, X, Y, L₁, L₂, Ar₁, Ar₂, R₁ and R₂ are as defined in Chemical Formula 1. Further, in Reaction Scheme 1, Z is halogen, preferably chloro.

The Reaction Scheme 1 is a Suzuki coupling reaction which is performed in the presence of a palladium catalyst and a base. Further, the reactive group for the Suzuki coupling reaction can be modified as known in the art. Further, the above preparation method can be further embodied in the Preparation Examples described hereinafter.

(Organic Light Emitting Device)

Meanwhile, the present disclosure provides an organic light emitting device comprising a compound of Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer, and the like as the organic material layer. However, the structure of the organic light emitting device is not limited thereto, and it can include a smaller number of organic layers.

Further, the organic material layer can include a hole injection layer, a hole transport layer, or a layer for simultaneously performing hole injection and transport, wherein the hole injection layer, the hole transport layer, or the layer for simultaneously performing hole injection and transport includes the compound of Chemical Formula 1.

Further, the organic material layer can include a light emitting layer, wherein the light emitting layer can include the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure further comprising a hole injection layer and a hole transport layer between the first electrode and the light emitting layer, and an electron transport layer and an electron injection layer between the light emitting layer and the second electrode, in addition to the light emitting layer, as the organic material layer. However, the structure of the organic light emitting device is not limited thereto, and it can include a smaller number of organic layers or a larger number of organic layers.

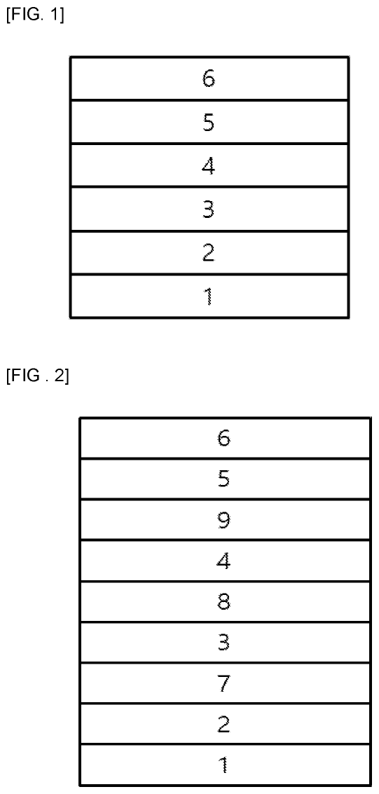

Further, the organic light emitting device according to the present disclosure can be a normal type organic light emitting device in which an anode, one or more organic material layers and a cathode are sequentially stacked on a substrate, wherein the first electrode is an anode, and the second electrode is a cathode. Further, the organic light emitting device according to the present disclosure can be an inverted type organic light emitting device in which a cathode, one or more organic material layers and an anode are sequentially stacked on a substrate, wherein the first electrode is a cathode and the second electrode is an anode. For example, the structure of the organic light emitting device according to an embodiment of the present disclosure is illustrated in FIGS. 1 and 2.

FIG. 1 shows an example of an organic light emitting device comprising a substrate 1, an anode 2, a hole transport layer 3, a light emitting layer 4, an electron injection and transport layer 5, and a cathode 6. In such a structure, the compound of Chemical Formula 1 can be included in the hole transport layer.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6. In such a structure, the compound of Chemical Formula 1 can be included in the hole injection layer, the hole transport layer, or the electron blocking layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer, and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes up to the light emitting layer. The hole transport material is suitably a material having large mobility to the holes, which can receive holes from the anode or the hole injection layer and transfer the holes to the light emitting layer. Examples of such hole transport material can include the compound of Chemical Formula 1, or an arylamine-based organic material, a conductive polymer, a block copolymer in which a conjugate portion and a non-conjugate portion are present together, and the like, but are not limited thereto.

The electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include the compound of Chemical Formula 1, or an arylamine-based organic material or the like, but is not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples thereof include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

The light emitting layer can include a host material and a dopant material as described above. The host material can further include a fused aromatic ring derivative, a heterocyclic-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

The dopant material can be an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer refers to a layer which is formed on the light emitting layer, preferably provided in contact with the light emitting layer, and serves to adjust the electron mobility, prevent excessive movement of holes, and increase the probability of hole-electron coupling, thereby improving the efficiency of the organic light emitting device. The hole blocking layer includes a hole blocking material, and examples of such hole blocking material can include a compound having an electron withdrawing group introduced therein, such as azine derivatives including triazine, triazole derivatives, oxadiazole derivatives, phenanthroline derivatives, phosphine oxide derivatives, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: a Al complex of 8-hydroxyquinoline; a complex including Alq₃; an organic radical compound; a hydroxyflavone-metal complex, a triazine derivative, and the like, but are not limited thereto. Alternatively, it can be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure can be a bottom emission device, a top emission device, or a double-sided light emitting device, and in particular, can be a bottom emission device that requires relatively high luminous efficiency.

In addition, the compound of Chemical Formula 1 can be included in an organic solar cell or an organic transistor in addition to an organic light emitting device.

### PREPARATION EXAMPLES

### Preparation Example 1: Preparation of Compound Sub 1-2

First, Compound A-1 was prepared.

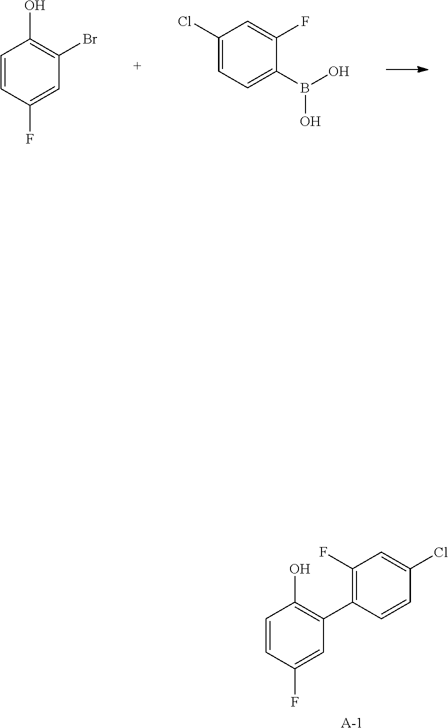 [SMILES: Oc1ccc(F)cc1Br ; OB(O)c1ccc(Cl)cc1F ; Oc1ccc(F)cc1-c1ccc(Cl)cc1F ; "A-1"]

2-Bromo-4-fluorophenol (100 g, 526.5 mmol) and (4-chloro-2-fluorophenyl)boronic acid (91.6 g, 526.5 mmol) were added to tetrahydrofuran (2000 ml) under a nitrogen atmosphere, and the mixture was stirred and refluxed. Then, potassium carbonate (218.3 g, 1579.4 mmol) was dissolved in water (218 ml), added thereto, sufficiently stirred and then tetrakis(triphenylphosphine)palladium(0) (18.2 g, 15.8 mmol) was added. After reacting for 3 hours, the reaction mixture was cooled to room temperature, and the resulting solid was filtered. The solid was added to and dissolved in chloroform (6739 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a while solid Compound A-1 (78.2 g, yield: 58%, MS: [M+H]⁺=257).

Next, Compound A-2 was prepared.

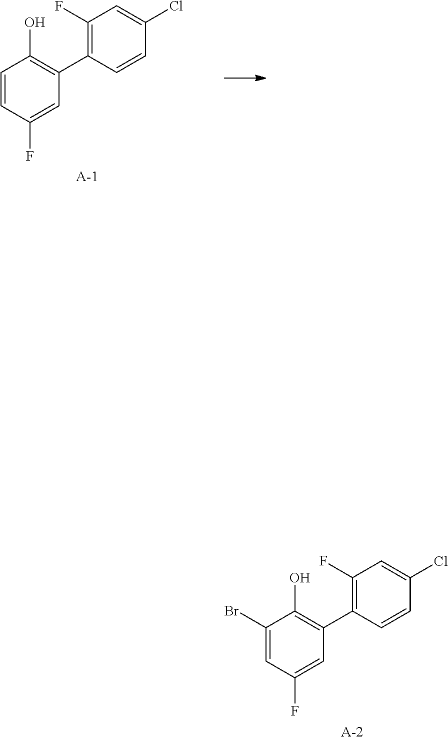 [SMILES: Oc1ccc(F)cc1-c1ccc(Cl)cc1F ; "A-1" ; Oc1c(Br)cc(F)cc1-c1ccc(Cl)cc1F ; "A-2"]

Compound A-1 (70 g, 273.4 mmol) and N-bromosuccinimide (48.7 g, 273.4 mmol) were added to chloroform (350 ml) under a nitrogen atmosphere, and the mixture was stirred and cooled to 0° C. After reacting for 5 hours, the reaction mixture was cooled to room temperature, and then water was added thereto. Then, the organic layer and the aqueous layer were separated and then the organic layer was concentrated. This was again added to and dissolved in chloroform (913 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a while solid Compound A-2 (58.4 g, yield: 64%, MS: [M+H]⁺=334.9).

Next, Compound A-3 was prepared.

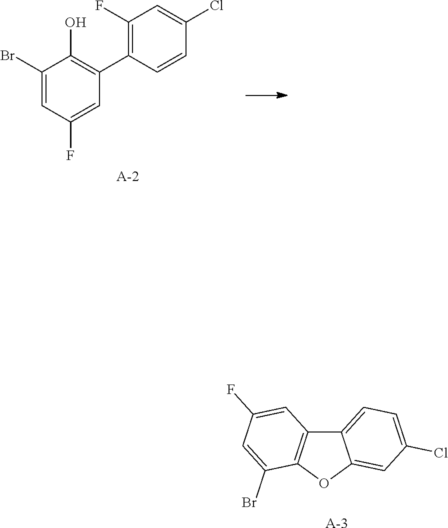

Under a nitrogen atmosphere, Compound A-2 (50 g, 149.7 mmol) was added to dimethylformamide (250 ml), potassium carbonate was added, and then the mixture was stirred and heated to 140° C. After reacting for 6 hours, the reaction mixture was cooled to room temperature, and then water added thereto. Then, the resulting solid was filtered. This was again added to and dissolved in chloroform (380 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a while solid Compound A-3 (22.8 g, yield: 60%, MS: [M+H]⁺=255).

Next, Compound A-4 was prepared.

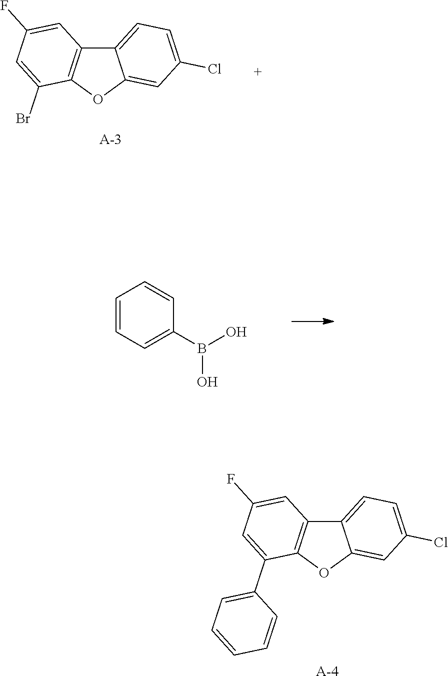

Compound A-3 (20 g, 67.1 mmol) and phenylboronic acid (8.2 g, 67.1 mmol) were added to tetrahydrofuran (400 ml) under a nitrogen atmosphere, and the mixture was stirred and refluxed. Then, potassium carbonate (27.8 g, 201.4 mmol) was dissolved in water (28 ml), added thereto, sufficiently stirred, and then tetrakis(triphenylphosphine)palladium(0) (2.3 g, 2 mmol) was added. After reacting for 3 hours, the reaction mixture was cooled to room temperature, the organic layer and the aqueous layer were separated and then the organic layer was distilled. This was again added to and dissolved in chloroform (397 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a while solid Compound A-4 (14.3 g, yield: 72%, MS: [M+H]⁺=297).

Next, Compound sub 1-1 was prepared.

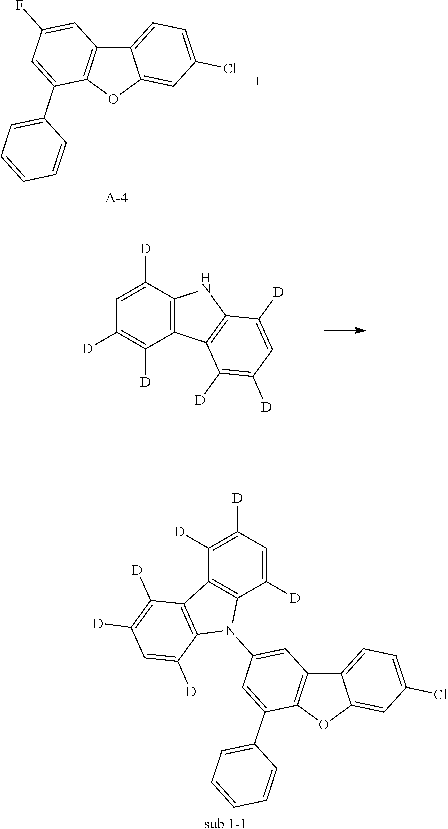

Compound A-4 (15 g, 50.7 mmol) and 9H-carbazole-1,3,4,5,6,8-D6 (8.8 g, 50.7 mmol) were added to dimethylformamide (300 ml) under a nitrogen atmosphere, and the mixture was stirred and refluxed. Then, potassium triphosphate (32.3 g, 152 mmol) was added thereto and then sufficiently stirred. After reacting for 3 hours, the reaction mixture was cooled to room temperature, the organic layer was filtered to remove salts, and then the filtered organic layer was distilled. This was again added to and dissolved in chloroform (228 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a while solid Compound sub 1-1 (11.6 g, yield: 51%, MS: [M+H]⁺=450.2).

Next, Compound sub 1-2 was prepared.

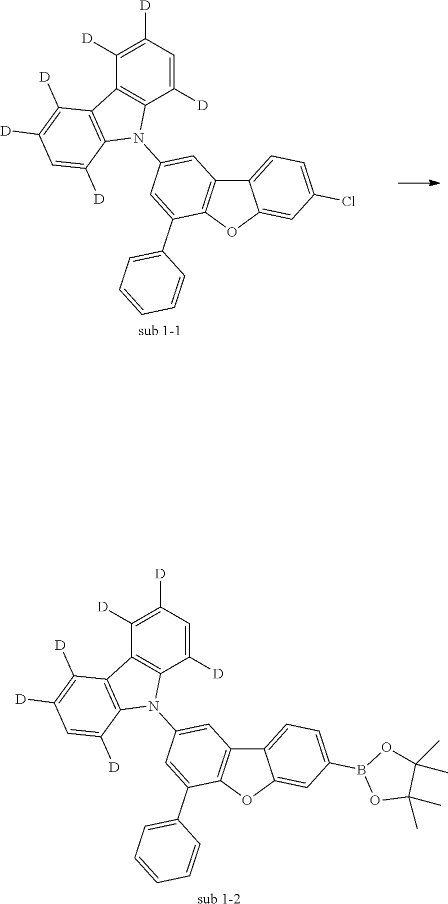

Compound sub 1-1 (15 g, 33.4 mmol) and bis(pinacolato)diboron (9.3 g, 36.7 mmol) were added to dioxane (300 ml) under a nitrogen atmosphere, and the mixture was stirred and refluxed. Then, potassium acetate (9.6 g, 100.2 mmol) was added thereto, sufficiently stirred, and then bis(dibenzylideneacetone)palladium(0) (0.6 g, 1 mmol) and tricyclohexylphosphine (0.6 g, 2 mmol) were added. After reacting for 4 hours, the reaction mixture was cooled to room temperature, the organic layer was filtered to remove salts, and then the filtered organic layer was distilled. This was again added to and dissolved in chloroform (181 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethanol to give a while solid Compound sub 1-2 (15 g, yield: 83%, MS: [M+H]⁺=542.3).

### Preparation Example 2: Preparation of Compound Sub 2-2

First, Compound sub 2-1 was prepared.

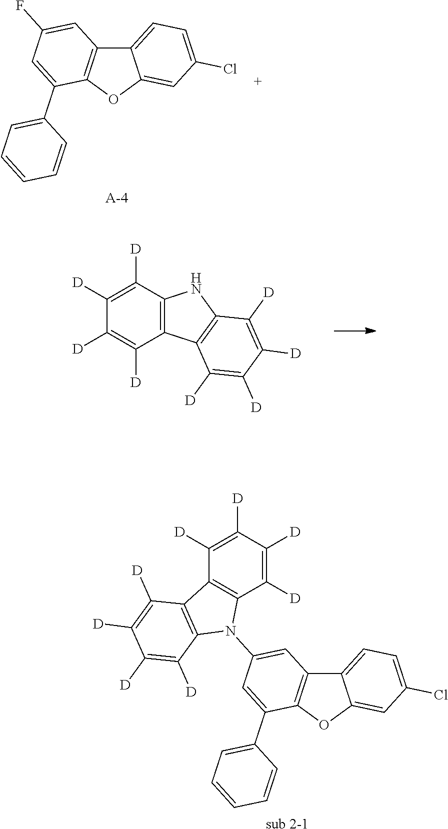

Compound A-4 (15 g, 50.7 mmol) and 9H-carbazole-1,2,3,4,5,6,7,8-D8 (8.9 g, 50.7 mmol) were added to dimethylformamide (300 ml) under a nitrogen atmosphere, and the mixture was stirred and refluxed. Then, potassium triphosphate (32.3 g, 152 mmol) was added thereto and then sufficiently stirred. After reacting for 7 hours, the reaction mixture was cooled to room temperature, the organic layer was filtered to remove salts, and then the filtered organic layer was distilled. This was again added to and dissolved in chloroform (229 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a yellow solid Compound sub 2-1 (11.7 g, yield: 51%, MS: [M+H]⁺=452.2).

Next, Compound sub 2-2 was prepared.

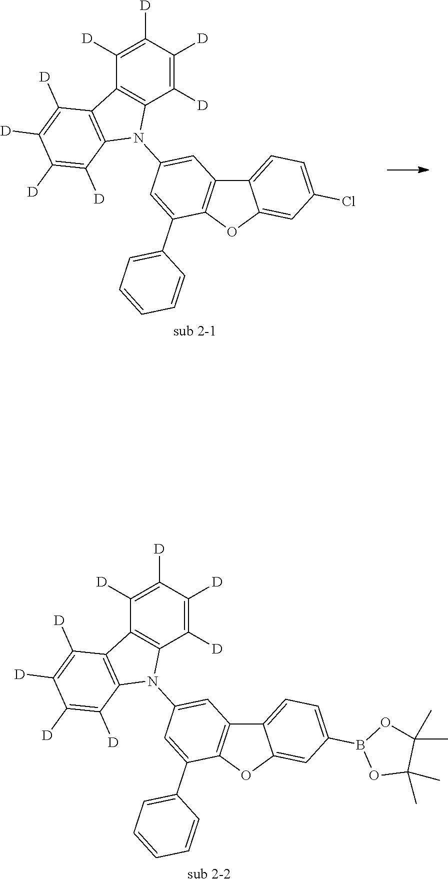

Compound sub 2-1 (15 g, 33.2 mmol) and bis(pinacolato)diboron (9.3 g, 36.6 mmol) were added to dioxane (300 ml) under a nitrogen atmosphere, and the mixture was stirred and refluxed. Then, potassium acetate (9.6 g, 99.7 mmol) was added thereto, sufficiently stirred, and then bis(dibenzylideneacetone)palladium(0) (0.6 g, 1 mmol) and tricyclohexylphosphine (0.6 g, 2 mmol) were added. After reacting for 3 hours, the reaction mixture was cooled to room temperature, the organic layer was filtered to remove salts, and then the filtered organic layer was distilled. This was again added to and dissolved in chloroform (181 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethanol to give a while solid Compound sub 2-2 (10.7 g, yield: 59%, MS: [M+H]⁺=544.3).

### Preparation Example 3: Preparation of Compound Sub 3-2

First, Compound B-1 was prepared.

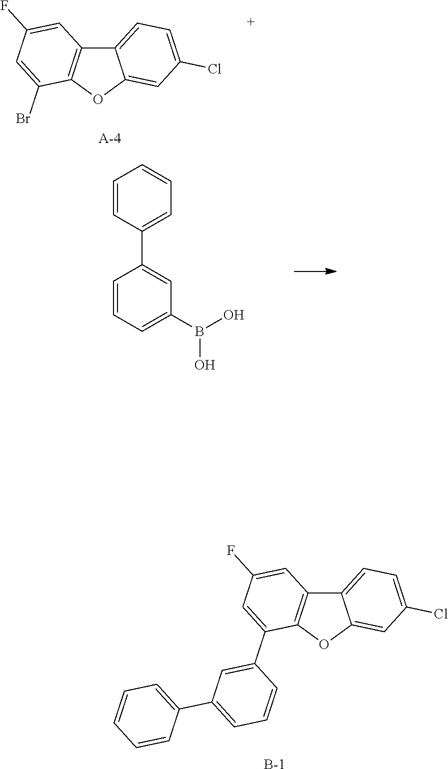

Compound A-3 (20 g, 67.1 mmol) and [1,1′-biphenyl]-3-ylboronic acid (13.3 g, 67.1 mmol) were added to tetrahydrofuran (400 ml) under a nitrogen atmosphere, and the mixture was stirred and refluxed. Then, potassium carbonate (27.8 g, 201.4 mmol) was dissolved in water (28 ml), added thereto, sufficiently stirred, and then tetrakis(triphenylphosphine)palladium(0) (2.3 g, 2 mmol) was added. After reacting for 1 hour, the reaction mixture was cooled to room temperature, the organic layer and the aqueous layer were separated and then the organic layer was distilled. This was again added to and dissolved in chloroform (500 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow solid Compound B-1 (16.5 g, yield: 66%, MS: [M+H]⁺=373.1).

Next, Compound sub 3-1 was prepared.

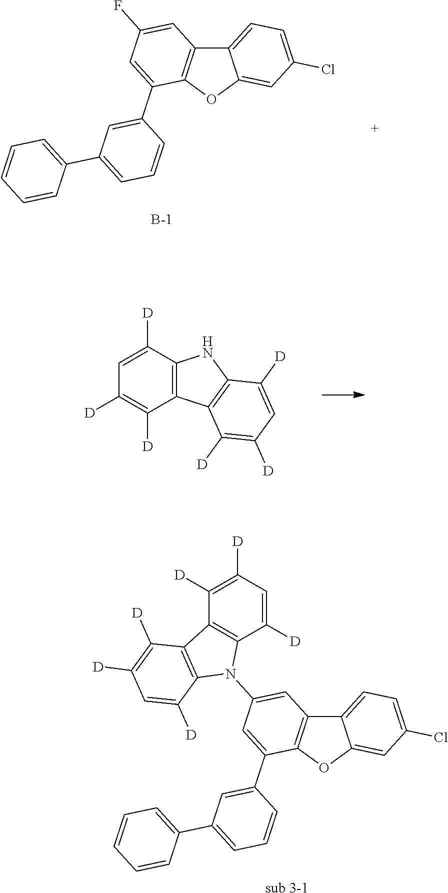

Compound B-1 (15 g, 40.3 mmol) and 9H-carbazole-1,3,4,5,6,8-D6 (7 g, 40.3 mmol) were added to dimethylformamide (300 ml) under a nitrogen atmosphere, and the mixture was stirred and refluxed. Then, potassium triphosphate (25.7 g, 120.9 mmol) was added thereto and sufficiently stirred. After reacting for 3 hours, the reaction mixture was cooled to room temperature, the organic layer was filtered to remove salts, and then the filtered organic layer was distilled. This was again added to and dissolved in chloroform (213 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a yellow solid Compound sub 3-1 (14.5 g, yield: 68%, MS: [M+H]⁺=529.2).

Next, Compound sub 3-2 was prepared.

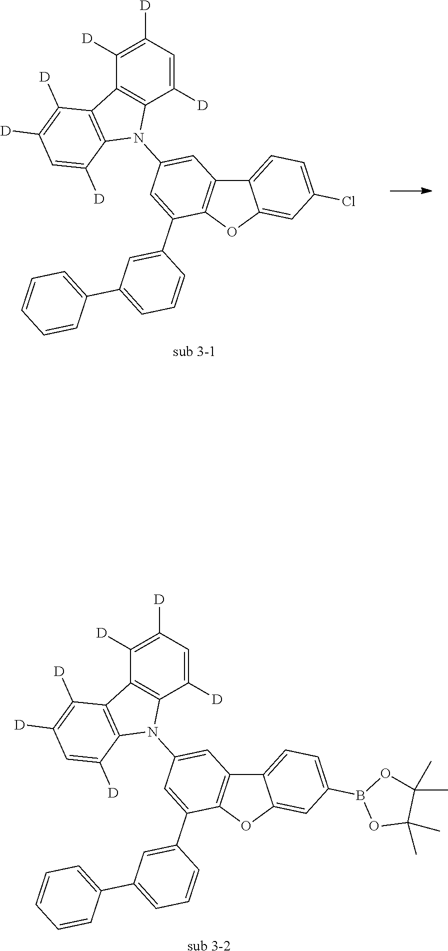

Compound sub 3-1 (15 g, 28.6 mmol) and bis(pinacolato)diboron (8 g, 31.4 mmol) were added to 300 ml of dioxane under a nitrogen atmosphere, and the mixture was stirred and refluxed. Then, potassium acetate (8.2 g, 85.7 mmol) was added thereto, sufficiently stirred, and then bis(dibenzylideneacetone)palladium(0) (0.5 g, 0.9 mmol) and tricyclohexylphosphine (0.5 g, 1.7 mmol) were added. After reacting for 5 hours, the reaction mixture was cooled to room temperature, the organic layer was filtered to remove salts, and then the filtered organic layer was distilled. This was again added to and dissolved in chloroform (176 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethanol to give a gray solid Compound sub 3-2 (8.8 g, yield: 50%, MS: [M+H]⁺=618.3).

### Preparation Example 4: Preparation of Compound Sub 4-2

First, Compound C-1 was prepared.

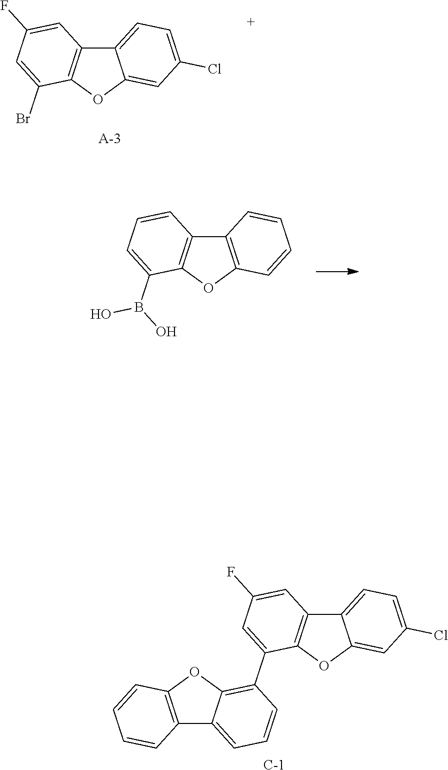

Compound A-3 (20 g, 67.1 mmol) and dibenzo[b,d]furan-4-ylboronic acid (14.2 g, 67.1 mmol) were added to tetrahydrofuran (400 ml) under a nitrogen atmosphere, and the mixture was stirred and refluxed. Then, potassium carbonate (27.8 g, 201.4 mmol) was dissolved in water (28 ml), added thereto, sufficiently stirred, and then tetrakis(triphenylphosphine)palladium(0) (2.3 g, 2 mmol) was added. After reacting for 3 hours, the reaction mixture was cooled to room temperature, the organic layer and the aqueous layer were separated, and then the organic layer was distilled. This was again added to and dissolved in chloroform (518 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a while solid Compound C-1 (14.8 g, yield: 57%, MS: [M+H]⁺=387.1).

Next, Compound sub 4-1 was prepared.

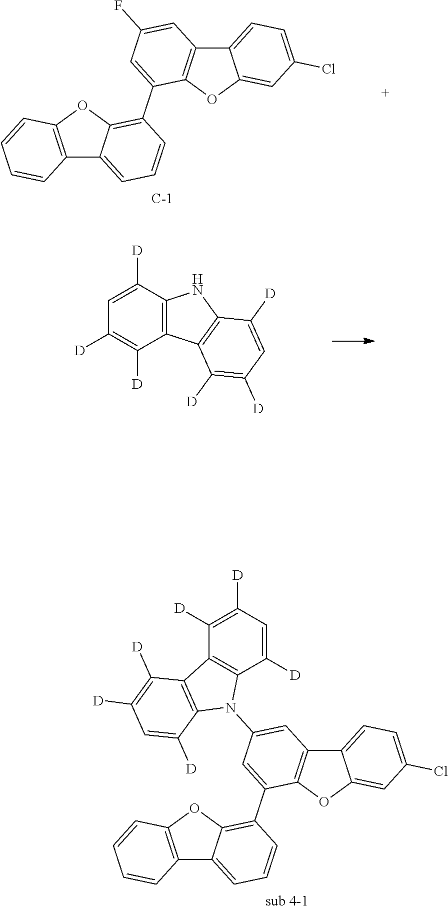

Compound C-1 (15 g, 38.9 mmol) and 9H-carbazole-1,3,4,5,6,8-D6 (6.7 g, 38.9 mmol) were added to dimethylformamide (300 ml) under a nitrogen atmosphere, and the mixture was stirred and refluxed. Then, potassium triphosphate (24.7 g, 116.6 mmol) was added thereto and sufficiently stirred. After reacting for 5 hours, the reaction mixture was cooled to room temperature, the organic layer was filtered to remove salts, and then the filtered organic layer was distilled. This was again added to and dissolved in chloroform (209 mL), washed twice with water, and then the organic layer was separated, anhydrous magnesium sulfate was added thereto, stirred, and then filtered, and the filtrate was distilled under reduced pressure. The concentrated compound was purified through a silica column using chloroform and ethyl acetate to give a while solid Compound sub 4-1 (10.9 g, yield: 52%, MS: [M+H]⁺=540.2).

Next, Compound sub 4-2 was prepared.

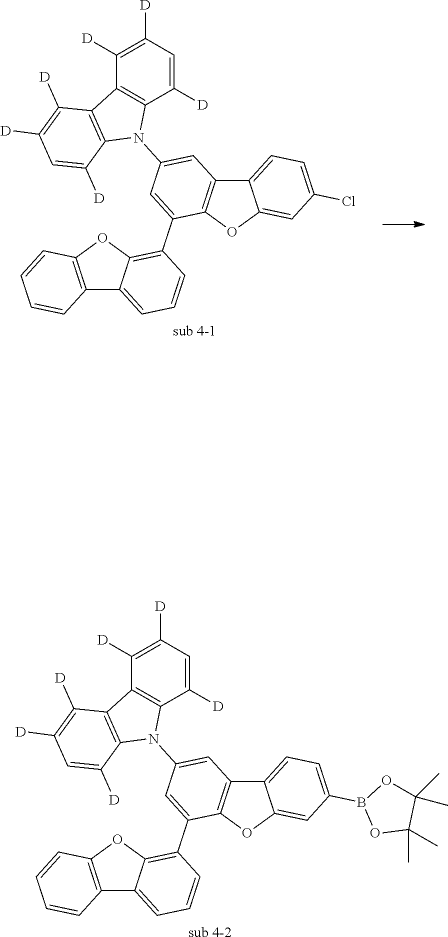

Compound sub 4-1 (15 g, 27.8 mmol) and bis(pinacolato)diboron (7.8 g, 30.6 mmol) were added to dioxane (300 ml) under a nitrogen atmosphere, and the mixture was stirred and refluxed. Then, potassium acetate (8 g, 83.5 mmol) was added thereto, sufficiently stirred, and then bis(dibenzylideneacetone)palladium(0) (0.5 g, 0.8 mmol) and tricyclohexylphosphine (0.5 g, 1.7 mmol) were added. After reacting for 6 hours, the reaction mixture was cooled to room temperature, the organic layer was filtered to remove salts, and then the filtered organic layer was distilled. This was again added to and dissolved in chloroform (176 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a while solid Compound sub 4-2 (12.3 g, yield: 70%, MS: [M+H]⁺=632.3).

### EXAMPLES

### Example 1: Synthesis of Compound 1

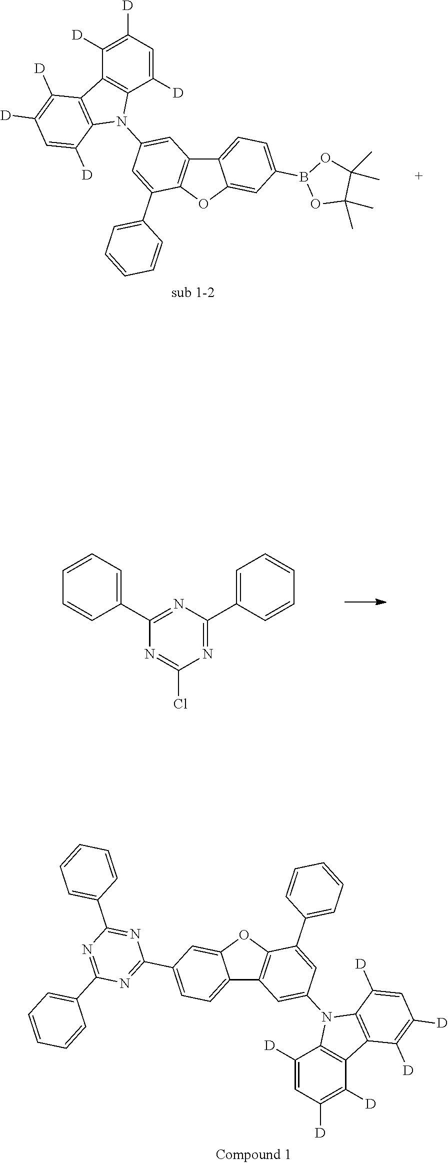

Compound sub 1-2 (10 g, 18.5 mmol) and 2-chloro-4,6-diphenyl-1,3,5-triazine (4.9 g, 18.5 mmol) were added to tetrahydrofuran (200 ml) under a nitrogen atmosphere, and the mixture was stirred and refluxed. Then, potassium acetate (7.7 g, 55.4 mmol) was dissolved in water (8 ml), added thereto, sufficiently stirred, and then tetrakis(triphenylphosphine)palladium (0.6 g, 0.6 mmol) was added. After reacting for 3 hours, the reaction mixture was cooled to room temperature, the organic layer and the aqueous layer were separated and then the organic layer was distilled. This was again added to and dissolved in chloroform (238 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 1 (7.7 g, yield: 65%, MS: [M+H]⁺=646.3).

### Example 2: Synthesis of Compound 2

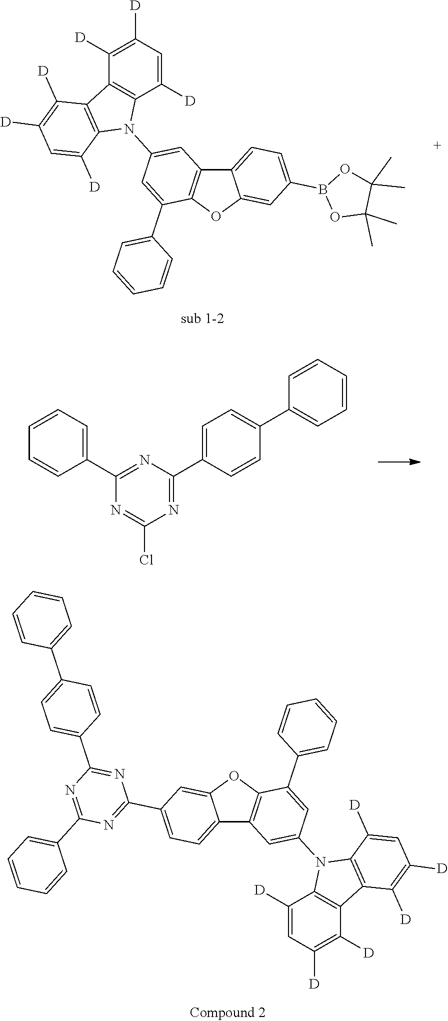 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2ccc(-c3ccccc3)cc2)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6ccccc6)cc5)n4)ccc3c2c1]

Compound sub 1-2 (10 g, 18.5 mmol) and 2-([1,1′-biphenyl]-4-yl)-4-chloro-6-phenyl-1,3,5-triazine (6.3 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 3 hours, the reaction mixture was cooled to room temperature, the organic layer and the aqueous layer were separated and then the organic layer was distilled. This was again added to and dissolved in chloroform (267 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 2 (8.1 g, yield: 61%, MS: [M+H]⁺=723.3).

### Example 3: Synthesis of Compound 3

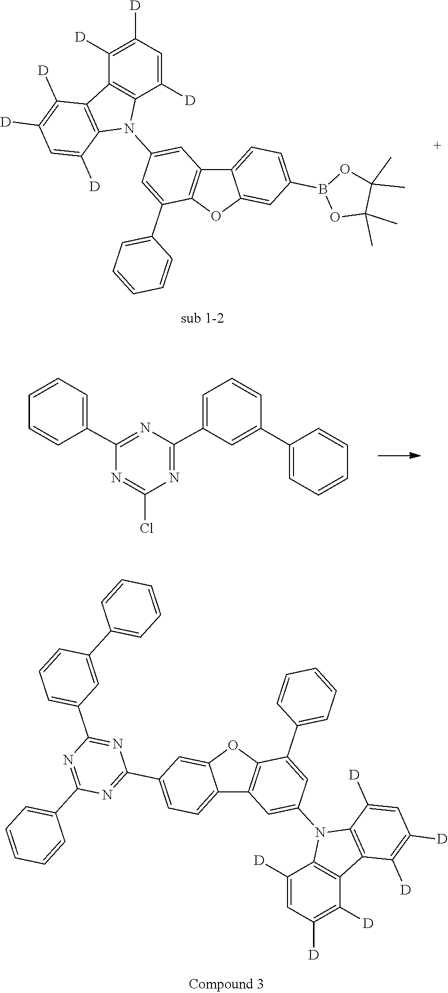 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2cccc(-c3ccccc3)c2)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc(-c6ccccc6)c5)n4)ccc3c2c1]

Compound sub 1-2 (10 g, 18.5 mmol) and 2-([1,1′-biphenyl]-3-yl)-4-chloro-6-phenyl-1,3,5-triazine (6.3 g, 18.5 mmol) were added to tetrahydrofuran (208 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)paladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (267 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 3 (8.3 g, yield: 62%, MS: [M+H]⁺=723.3).

### Example 4: Synthesis of Compound 4

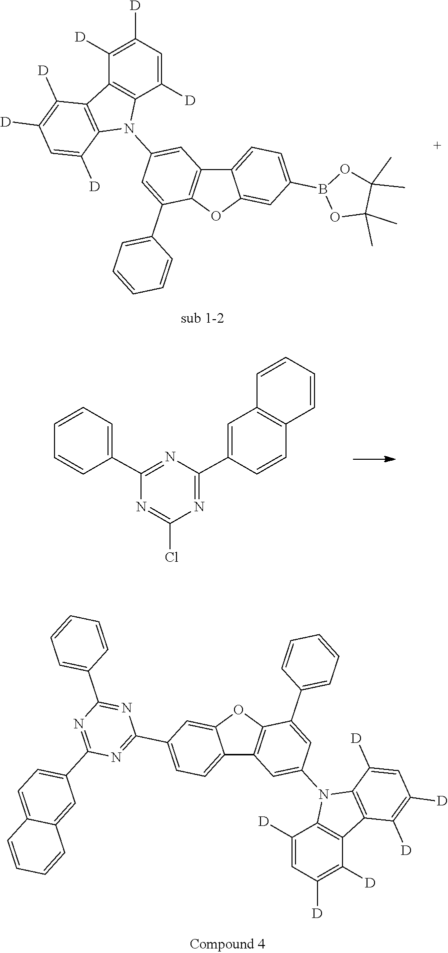 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2ccc3ccccc3c2)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6ccccc6c5)n4)ccc3c2c1]

Compound sub 1-2 (10 g, 18.5 mmol) and 2-chloro-4-(naphthalen-2-yl)-6-phenyl-1,3,5-triazine (5.9 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (257 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 4 (9 g, yield: 70%, MS: [M+H]⁺=697.3).

### Example 5: Synthesis of Compound 5

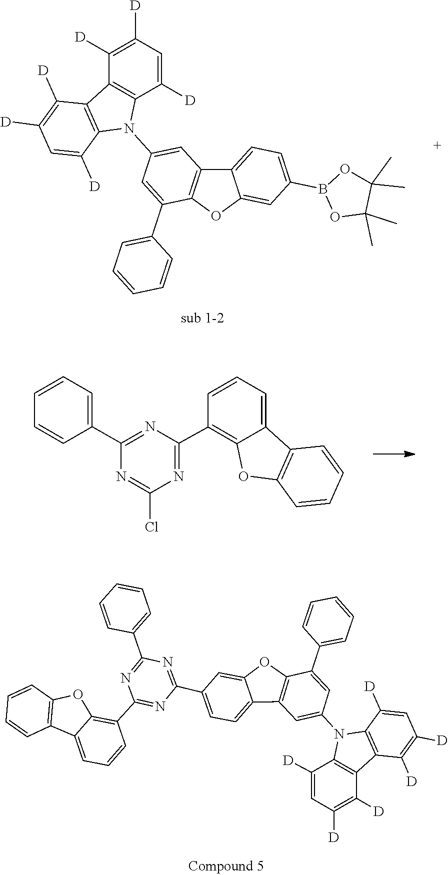 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2cccc3c2oc2ccccc23)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5oc5ccccc56)n4)ccc3c2c1]

Compound sub 1-2 (10 g, 18.5 mmol) and 2-chloro-4-(dibenzo[b,d]furan-4-yl)-6-phenyl-1,3,5-triazine (6.6 g, 18.5 mmol) were added to tetrahydrofuran (208 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (272 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 5 (7.8 g, yield: 57%, MS: [M+H]⁺=737.3).

### Example 6: Synthesis of Compound 6

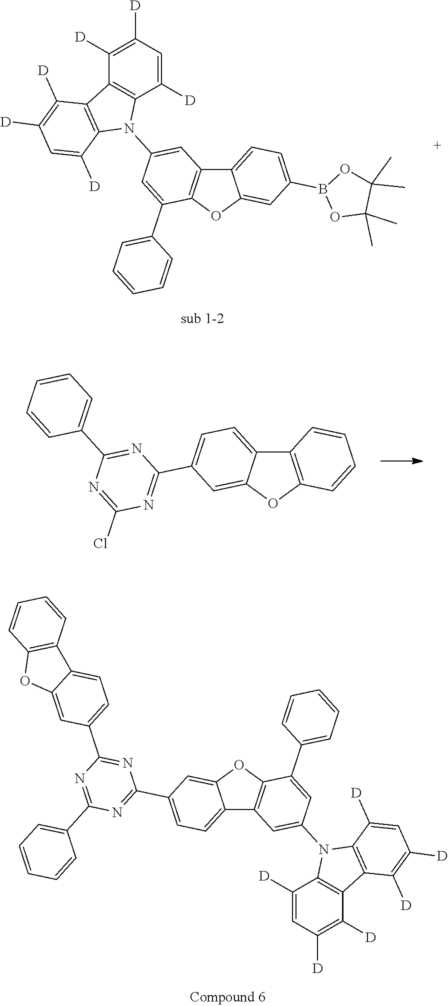 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2ccc3c(c2)oc2ccccc23)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)oc5ccccc56)n4)ccc3c2c1]

Compound sub 1-2 (10 g, 18.5 mmol) and 2-chloro-4-(dibenzo[b,d]furan-3-yl)-6-phenyl-1,3,5-triazine (6.6 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (272 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 6 (7.2 g, yield: 53%, MS: [M+H]⁺=737.3).

### Example 7: Synthesis of Compound 7

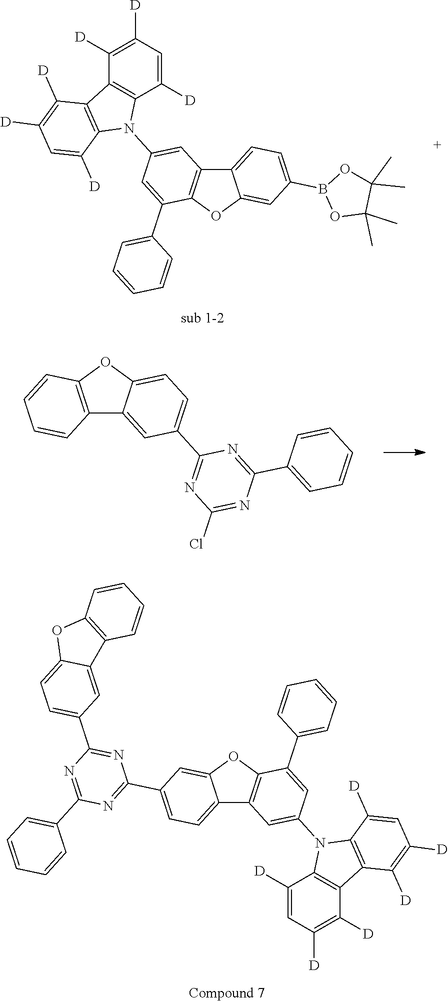 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2ccc3oc4ccccc4c3c2)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6oc7ccccc7c6c5)n4)ccc3c2c1]

Compound sub 1-2 (10 g, 18.5 mmol) and 2-chloro-4-(dibenzo[b,d]furan-2-yl)-6-phenyl-1,3,5-triazine (6.6 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 1 hour, the reaction mixture was cooled to room temperature, the organic layer and the aqueous layer were separated and then the organic layer was distilled. This was again added to and dissolved in chloroform (272 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 7 (9.8 g, yield: 72%, MS: [M+H]⁺=737.3).

### Example 8: Synthesis of Compound 8

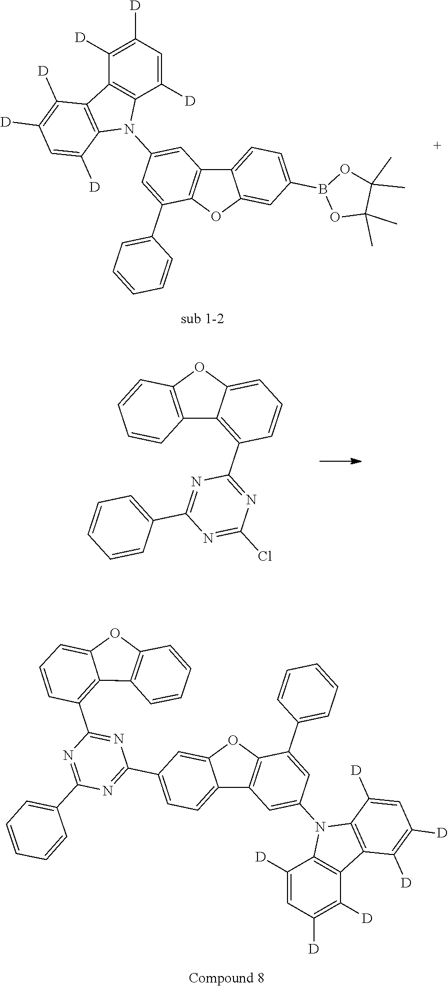 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2cccc3oc4ccccc4c23)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6oc7ccccc7c56)n4)ccc3c2c1]

Compound sub 1-2 (10 g, 18.5 mmol) and 2-chloro-4-(dibenzo[b,d]furan-1-yl)-6-phenyl-1,3,5-triazine (6.6 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (272 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 8 (9.1 g, yield: 67%, MS: [M+H]⁺=737.3).

### Example 9: Synthesis of Compound 9

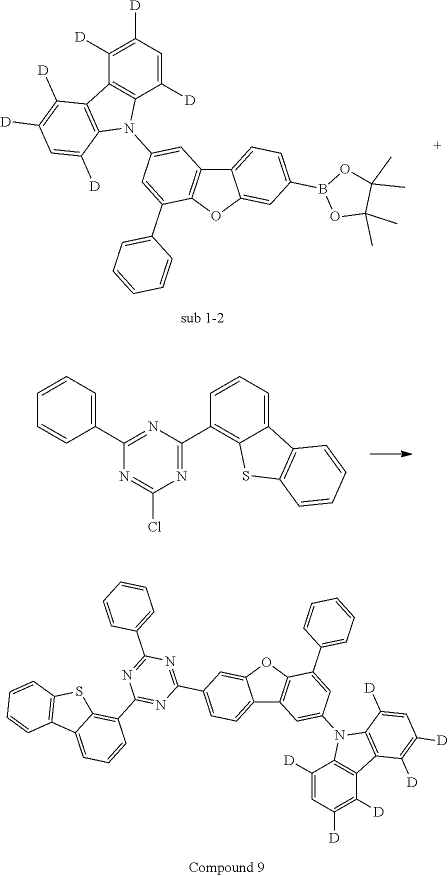 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-c2cccc3c2sc2ccccc23)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5sc5ccccc56)n4)ccc3c2c1]

Compound sub 1-2 (10 g, 18.5 mmol) and 2-chloro-4-(dibenzo[b,d]thiophen-4-yl)-6-phenyl-1,3,5-triazine (6.9 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (278 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 9 (8.5 g, yield: 61%, MS: [M+H]⁺=753.3).

### Example 10: Synthesis of Compound 10

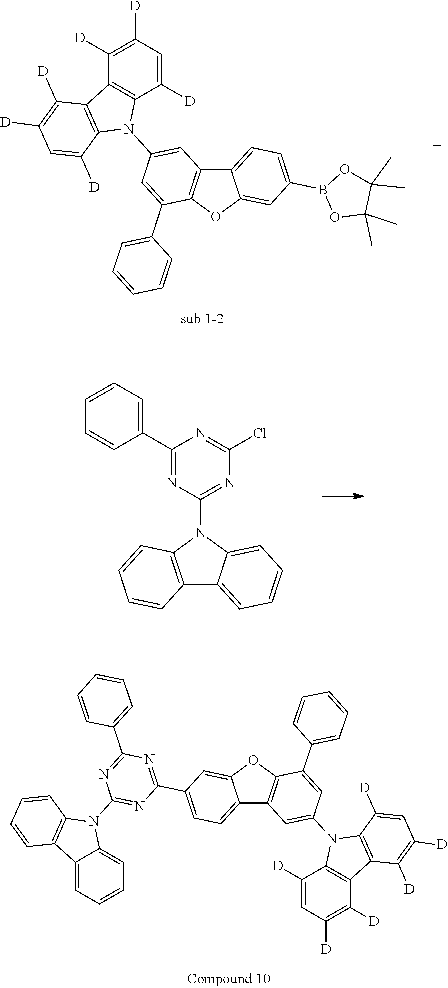 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; Clc1nc(-c2ccccc2)nc(-n2c3ccccc3c3ccccc32)n1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-n5c6ccccc6c6ccccc65)n4)ccc3c2c1]

Compound sub 1-2 (10 g, 18.5 mmol) and 9-(4-chloro-6-phenyl-1,3,5-triazin-2-yl)-9H-carbazole (6.6 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 1 hour, the reaction mixture was cooled to room temperature, the organic layer and the aqueous layer were separated and then the organic layer was distilled. This was again added to and dissolved in chloroform (272 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 10 (9.8 g, yield: 72%, MS: [M+H]⁺=736.3).

### Example 11: Synthesis of Compound 11

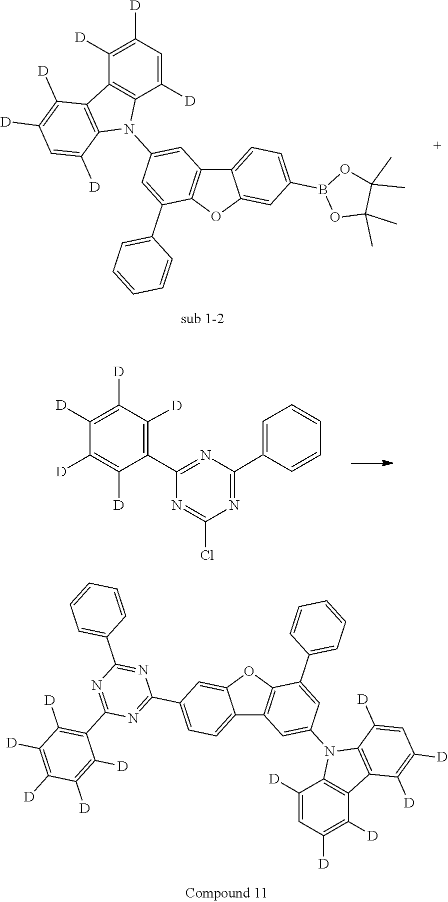 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; [2H]c1c([2H])c([2H])c(-c2nc(Cl)nc(-c3ccccc3)n2)c([2H])c1[2H] ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5c([2H])c([2H])c([2H])c([2H])c5[2H])n4)ccc3c2c1]

Compound sub 1-2 (10 g, 18.5 mmol) and 2-chloro-4-phenyl-6-(phenyl-D5)-1,3,5-triazine (5 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)paladium(0) (0.6 g, 0.6 mmol) was added. After reacting for 1 hour, the reaction mixture was cooled to room temperature, the organic layer and the aqueous layer were separated and then the organic layer was distilled. This was again added to and dissolved in chloroform (241 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 11 (6 g, yield: 50%, MS: [M+H]⁺=652.3).

### Example 12: Synthesis of Compound 12

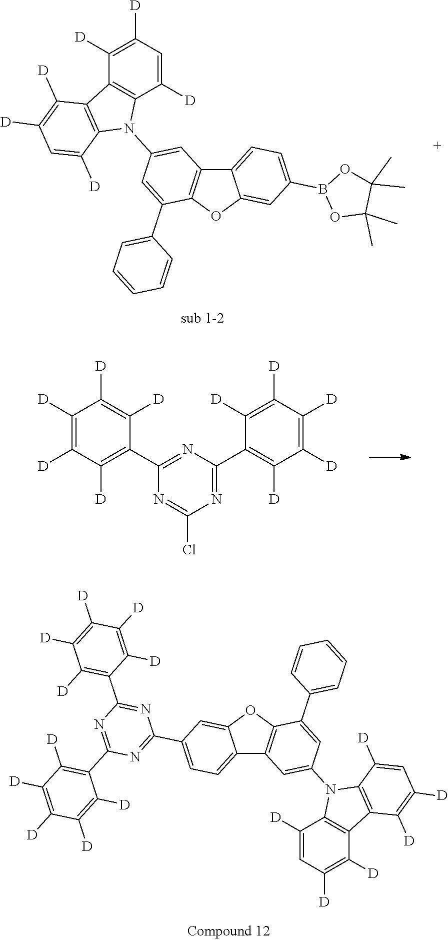 [SMILES: [2H]c1cc([2H])c2c(c1[2H])c1c([2H])c([2H])cc([2H])c1n2-c1cc(-c2ccccc2)c2oc3cc(B4OC(C)(C)C(C)(C)O4)ccc3c2c1 ; [2H]c1c([2H])c([2H])c(-c2nc(Cl)nc(-c3c([2H])c([2H])c([2H])c([2H])c3[2H])n2)c([2H])c1[2H] ; [2H]c1cc([2H])c2c(c1[2H])c1c([2H])c([2H])cc([2H])c1n2-c1cc(-c2ccccc2)c2oc3cc(-c4nc(-c5c([2H])c([2H])c([2H])c([2H])c5[2H])nc(-c5c([2H])c([2H])c([2H])c([2H])c5[2H])n4)ccc3c2c1]

Compound sub 1-2 (10 g, 18.5 mmol) and 2-chloro-4,6-bis(phenyl-D5)-1,3,5-triazine (5.1 g, 18.5 mmol) were added to tetrahydrofuran (200 ml) under a nitrogen atmosphere, and the mixture was stirred and refluxed. Then, potassium carbonate (7.7 g, 55.4 mmol) was dissolved in water (8 ml), added thereto, sufficiently stirred, and then tetrakis(triphenylphosphine)palladium(0) (0.6 g, 0.6 mmol) was added. After reacting for 1 hour, the reaction mixture was cooled to room temperature, the organic layer and the aqueous layer were separated and then the organic layer was distilled. This was again added to and dissolved in chloroform (243 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 12 (7 g, yield: 58%, MS: [M+H]⁺=657.3).

### Example 13: Synthesis of Compound 13

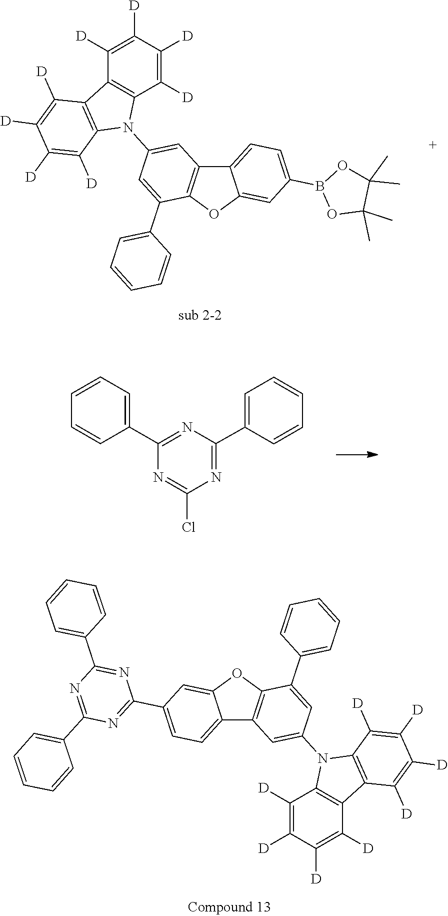 [SMILES: [2H]c1c([2H])c([2H])c2c(c1[2H])c1c([2H])c([2H])c([2H])c([2H])c1n2-c1cc(-c2ccccc2)c2oc3cc(B4OC(C)(C)C(C)(C)O4)ccc3c2c1 ; Clc1nc(-c2ccccc2)nc(-c2ccccc2)n1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1]

Compound sub 2-2 (10 g, 18.4 mmol) and 2-chloro-4,6-diphenyl-1,3,5-triazine (4.9 g, 18.4 mmol) were added to tetrahydrofuran (200 ml) under a nitrogen atmosphere, and the mixture was stirred and refluxed. Then, potassium carbonate (7.6 g, 55.2 mmol) was dissolved in water (8 ml), added thereto, sufficiently stirred, and then tetrakis(triphenylphosphine)palladium(0) (0.6 g, 0.6 mmol) was added. After reacting for 1 hour, the reaction mixture was cooled to room temperature, the organic layer and the aqueous layer were separated and then the organic layer was distilled. This was again added to and dissolved in chloroform (239 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 13 (9.1 g, yield: 76%, MS: [M+H]⁺=649.3).

### Example 14: Synthesis of Compound 14

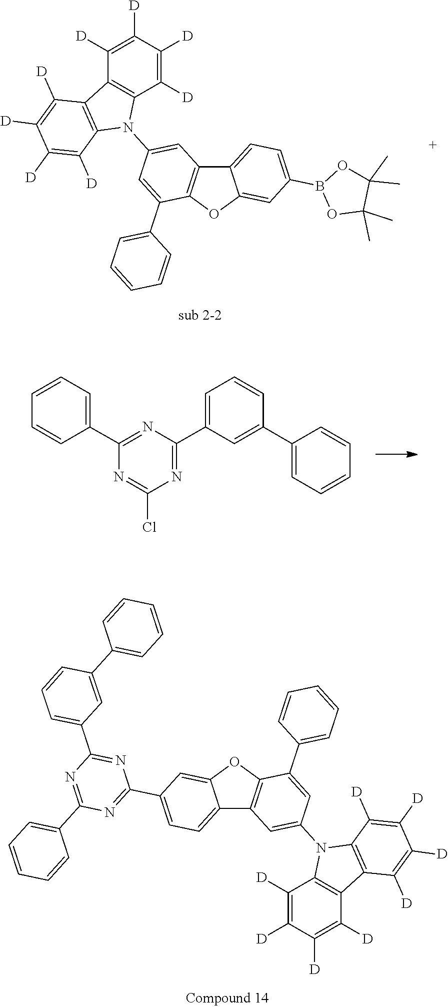 [SMILES: [2H]c1c([2H])c([2H])c2c(c1[2H])c1c([2H])c([2H])c([2H])c([2H])c1n2-c1cc(-c2ccccc2)c2oc3cc(B4OC(C)(C)C(C)(C)O4)ccc3c2c1 ; Clc1nc(-c2ccccc2)nc(-c2cccc(-c3ccccc3)c2)n1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5cccc(-c6ccccc6)c5)n4)ccc3c2c1]

Compound sub 2-2 (10 g, 18.4 mmol) and 2-([1,1′-biphenyl]-3-yl)-4-chloro-6-phenyl-1,3,5-triazine (6.3 g, 18.4 mmol) were added to tetrahydrofuran (200 ml) under a nitrogen atmosphere, and the mixture was stirred and refluxed. Then, potassium carbonate (7.6 g, 55.2 mmol) was dissolved in water (8 ml), added thereto, sufficiently stirred, and then tetrakis(triphenylphosphine)palladium(0) (0.6 g, 0.6 mmol) was added. After reacting for 2 hours, the reaction mixture was cooled to room temperature, the organic layer and the aqueous layer were separated and then the organic layer was distilled. This was again added to and dissolved in chloroform (267 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 14 (8.7 g, yield: 65%, MS: [M+H]⁺=725.3).

### Example 15: Synthesis of Compound 15

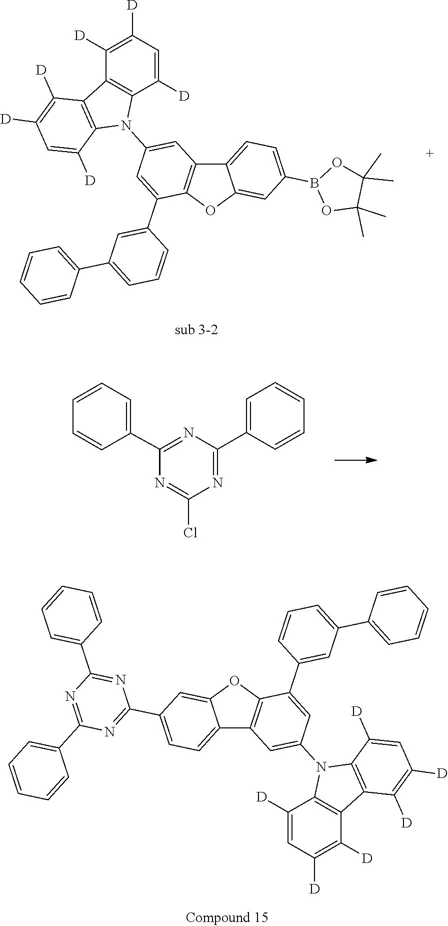 [SMILES: [2H]c1cc([2H])c2c(c1[2H])c1c([2H])c([2H])cc([2H])c1n2-c1cc(-c2cccc(-c3ccccc3)c2)c2oc3cc(B4OC(C)(C)C(C)(C)O4)ccc3c2c1 ; Clc1nc(-c2ccccc2)nc(-c2ccccc2)n1 ; [2H]c1cc([2H])c2c(c1[2H])c1c([2H])c([2H])cc([2H])c1n2-c1cc(-c2cccc(-c3ccccc3)c2)c2oc3cc(-c4nc(-c5ccccc5)nc(-c5ccccc5)n4)ccc3c2c1]

Compound sub 3-2 (10 g, 16.2 mmol) and 2-chloro-4,6-diphenyl-1,3,5-triazine (4.3 g, 16.2 mmol) were added to tetrahydrofuran (200 ml) under a nitrogen atmosphere, and the mixture was stirred and refluxed. Then, potassium carbonate (6.7 g, 48.6 mmol) was dissolved in water (7 ml), added thereto, sufficiently stirred, and then tetrakis(triphenylphosphine)-palladium(0) (0.6 g, 0.5 mmol) was added. After reacting for 1 hour, the reaction mixture was cooled to room temperature, the organic layer and the aqueous layer were separated and then the organic layer was distilled. This was again added to and dissolved in chloroform (234 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 15 (7.4 g, yield: 63%, MS: [M+H]⁺=723.3).

### Example 16: Synthesis of Compound 16

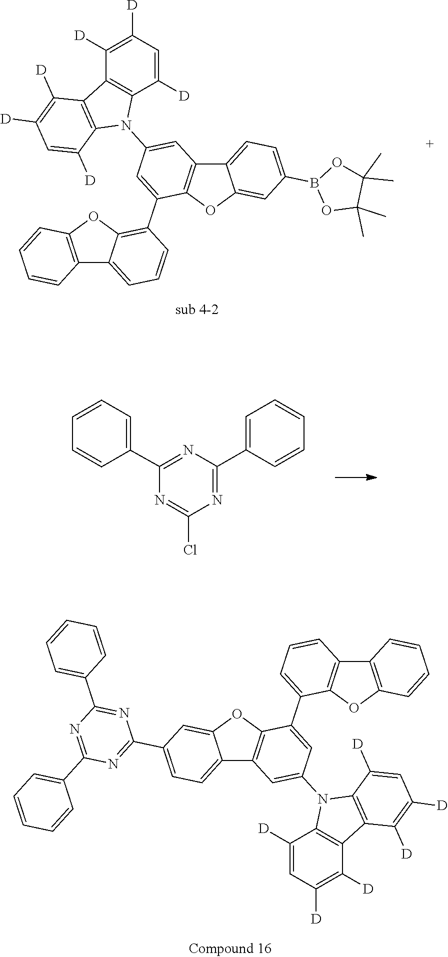 [SMILES: [2H]c1cc([2H])c2c(c1[2H])c1c([2H])c([2H])cc([2H])c1n2-c1cc(-c2cccc3c2oc2ccccc23)c2oc3cc(B4OC(C)(C)C(C)(C)O4)ccc3c2c1 ; Clc1nc(-c2ccccc2)nc(-c2ccccc2)n1 ; [2H]c1cc([2H])c2c(c1[2H])c1c([2H])c([2H])cc([2H])c1n2-c1cc(-c2cccc3c2oc2ccccc23)c2oc3cc(-c4nc(-c5ccccc5)nc(-c5ccccc5)n4)ccc3c2c1]

Compound sub 4-2 (10 g, 15.6 mmol) and 2-chloro-4,6-diphenyl-1,3,5-triazine (4.2 g, 15.6 mmol) were added to tetrahydrofuran (200 ml) under a nitrogen atmosphere, and the mixture was stirred and refluxed. Then, potassium carbonate (6.5 g, 46.8 mmol) was dissolved in water (6 ml), added thereto, sufficiently stirred, and then tetrakis(triphenylphosphine)-palladium(0) (0.5 g, 0.5 mmol) was added. After reacting for 3 hours, the reaction mixture was cooled to room temperature, the organic layer and the aqueous layer were separated and then the organic layer was distilled. This was again added to and dissolved in chloroform (230 mL), washed twice with water, and then the organic layer was separated, anhydrous magnesium sulfate was added thereto, stirred, and then filtered, and the filtrate was distilled under reduced pressure. The concentrated compound was recrystallized from chloroform and ethyl acetate to give a pale yellow Compound 16 (8.4 g, yield: 73%, MS: [M+H]⁺=737.3).

### EXPERIMENTAL EXAMPLE

### Experimental Example 1

A glass substrate on which a thin film of ITO (indium tin oxide) was coated in a thickness of 1,300 Å was put into distilled water containing a detergent dissolved therein and ultrasonically washed. In this case, the detergent used was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was thermally vacuum deposited to a thickness of 50 Å to form a hole injection layer. The following compound HT-1 was thermally vacuum deposited to a thickness of 250 Å on the hole injection layer to form a hole transport layer, and the following compound HT-2 was vacuum deposited to a thickness of 50 Å on the HT-1 deposited layer to form an electron blocking layer.

The compound 1 prepared in the Preparation Example 1, the following compound YGH-1, and the following phosphorescent dopant YGD-1 were co-deposited in a weight ratio of 44:44:12 on the HT-2 deposited layer to form a light emitting layer with a thickness of 400 Å. The following compound ET-1 was vacuum deposited to a thickness of 250 Å on the light emitting layer to form an electron transport layer, and the following compound ET-2 and Li were vacuum deposited in a weight ratio of 98:2 on the electron transport layer to form an electron injection layer with a thickness of 100 Å. Aluminum was deposited to a thickness of 1000 Å on the electron injection layer to form a cathode.

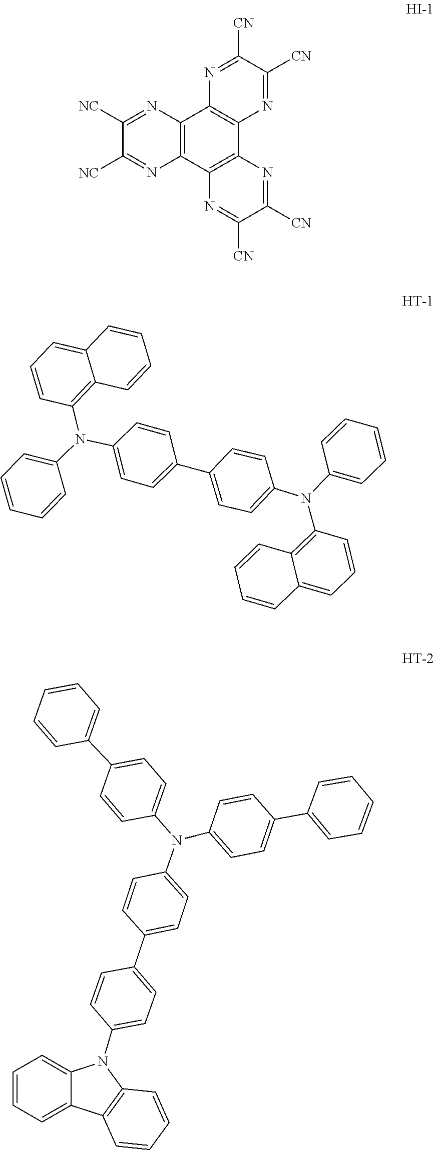 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccc(-n5c6ccccc6c6ccccc65)cc4)cc3)cc2)cc1]

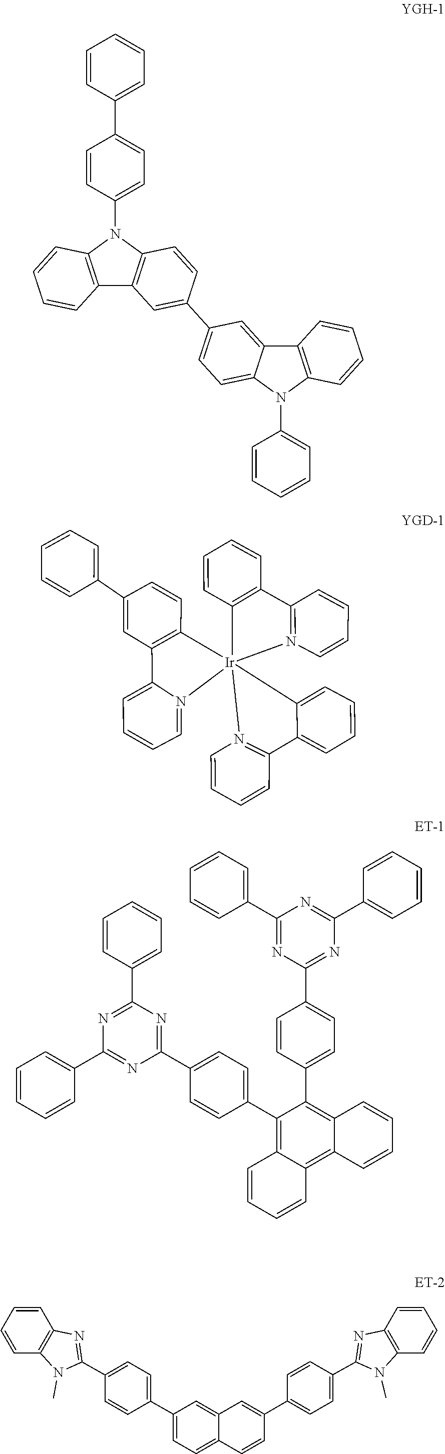

In the above-mentioned process, the vapor deposition rate of the organic material was maintained at 0.4 to 0.7 Å/sec, the deposition rate of aluminum was maintained at 2 Å/sec, and the degree of vacuum during the deposition was maintained at 1×10⁻⁷ to 5×10⁻⁸ torr.

### Experimental Examples 2 to 16

The organic light emitting devices were manufactured in the same manner as in Experimental Example 1, except that the compounds shown in Table 1 below were used instead of Compound 1 of Example 1 in Experimental Example 1.

### Comparative Experimental Examples 1 to 3

The organic light emitting devices were manufactured in the same manner as in Experimental Example 1, except that the compounds shown in Table 1 below were used instead of Compound 1 of Example 1 in Experimental Example 1. The compounds of CE1, CE2 and CE3 shown in Table 1 below are as follows.

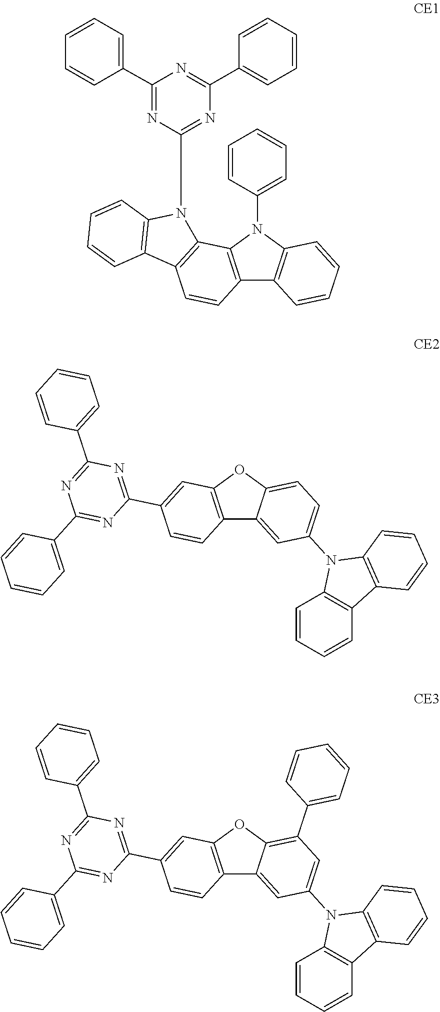 [SMILES: c1ccc(-c2nc(-c3ccccc3)nc(-n3c4ccccc4c4ccc5c6ccccc6n(-c6ccccc6)c5c43)n2)cc1 ; c1ccc(-c2nc(-c3ccccc3)nc(-c3ccc4c(c3)oc3ccc(-n5c6ccccc6c6ccccc65)cc34)n2)cc1 ; c1ccc(-c2nc(-c3ccccc3)nc(-c3ccc4c(c3)oc3c(-c5ccccc5)cc(-n5c6ccccc6c6ccccc65)cc34)n2)cc1]

For the organic light emitting devices manufactured in Experimental Examples and Comparative Experimental Examples, the voltage and efficiency were measured at a current density of 10 mA/cm², and the lifetime was measured at a current density of 50 mA/cm². The results are shown in Table 1 below. In this case, LT₉₅ means the time required for the luminance to be reduced to 95% of the initial luminance.

TABLE 1

| | | | Efficiency | Color | Lifetime (h) |
|---|---|---|---|---|---|
| | | Voltage (V) | (Cd/A) | coordinate | (LT₉₅ at |
| | Compound | (@10 mA/cm²) | (@10 mA/cm²) | (x, y) | 50 mA/cm²) |
| Experimental | Compound | 3.7 | 85 | 0.45, 0.55 | 300 |
| Example 1 | 1 | | | | |
| Experimental | Compound | 3.8 | 86 | 0.45, 0.55 | 320 |
| Example 2 | 2 | | | | |
| Experimental | Compound | 3.9 | 87 | 0.45, 0.55 | 340 |
| Example 3 | 3 | | | | |
| Experimental | Compound | 3.6 | 83 | 0.45, 0.55 | 280 |
| Example 4 | 4 | | | | |
| Experimental | Compound | 3.6 | 86 | 0.45, 0.55 | 325 |
| Example 5 | 5 | | | | |
| Experimental | Compound | 3.8 | 84 | 0.45, 0.55 | 420 |
| Example 6 | 6 | | | | |
| Experimental | Compound | 3.6 | 88 | 0.45, 0.55 | 250 |
| Example 7 | 7 | | | | |
| Experimental | Compound | 3.5 | 82 | 0.45, 0.55 | 345 |
| Example 8 | 8 | | | | |
| Experimental | Compound | 3.9 | 82 | 0.45, 0.55 | 445 |
| Example 9 | 9 | | | | |
| Experimental | Compound | 4.0 | 81 | 0.45, 0.55 | 415 |
| Example 10 | 10 | | | | |
| Experimental | Compound | 3.7 | 85 | 0.45, 0.55 | 335 |
| Example 11 | 11 | | | | |
| Experimental | Compound | 3.7 | 85 | 0.45, 0.55 | 370 |
| Example 12 | 12 | | | | |
| Experimental | Compound | 3.7 | 85 | 0.45, 0.55 | 350 |
| Example 13 | 13 | | | | |
| Experimental | Compound | 3.8 | 88 | 0.45, 0.55 | 368 |
| Example 14 | 14 | | | | |
| Experimental | Compound | 3.8 | 87 | 0.45, 0.55 | 395 |
| Example 15 | 15 | | | | |
| Experimental | Compound | 3.9 | 86 | 0.45, 0.55 | 365 |
| Example 16 | 16 | | | | |
| Comparative | CE 1 | 4.0 | 71 | 0.45, 0.55 | 120 |
| Experimental | | | | | |
| Example 1 | | | | | |
| Comparative | CE 2 | 3.9 | 82 | 0.45, 0.55 | 180 |
| Experimental | | | | | |
| Example 2 | | | | | |
| Comparative | CE 3 | 3.7 | 85 | 0.45, 0.55 | 200 |
| Experimental | | | | | |
| Example 3 | | | | | |

As shown in Table 1, it was confirmed that when the compound of the present disclosure was used as an organic light emitting layer material, it exhibited excellent characteristics in terms of efficiency and lifetime as compared with Comparative Experimental Examples. This is considered to be because triazine and carbazole groups are substituents of the dibenzofuran group, which is a core substituent, thereby increasing electronic stability. In particular, additional aryl group and carbazole group exhibits excellent properties of increasing the lifetime when substituted by at least one deuterium. This is also shown to have increased electronic stability.

[Description of Symbols]

| | |
|---|---|
| 1: substrate | 2: anode |
| 3: hole transport layer | 4: light emitting layer |
| 5: electron injection and transport layer | 6: cathode |
| 7: hole injection layer, | 8: electron blocking layer |
| 9: hole blocking layer | |

1. A compound of Chemical Formula 1: 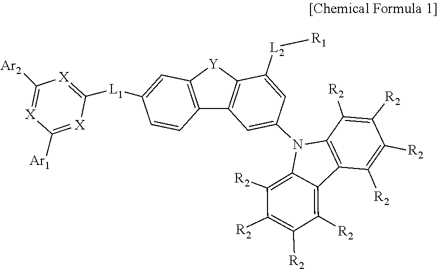
wherein, in Chemical Formula 1:
each X is independently N or CH, with two or more of the X being N;
Y is O or S;
L₁ is a direct bond or a substituted or unsubstituted C₆₋₆₀ arylene;
L₂ is a direct bond or a substituted or unsubstituted C₆₋₆₀ arylene;
Ar₁ and Ar₂ are each independently a substituted or unsubstituted C₆₋₆₀ aryl, or a substituted or unsubstituted C₆₋₆₀ heteroaryl containing any one or more heteroatoms selected from the group consisting of N, O and S,
R₁ is a substituted or unsubstituted C₆₋₆₀ aryl, or a substituted or unsubstituted C₆₋₆₀ heteroaryl containing any one or more heteroatoms selected from the group consisting of N, O and S; and
each R₂ is independently hydrogen or deuterium;
with the proviso that at least one of Ar₁, Ar₂ and R₁ is substituted with at least one deuterium, or at least one of R₂ is deuterium.

2. The compound of claim 1, wherein:
all X are N.

3. The compound of claim 1, wherein:
Y₁ is O.

4. The compound of claim 1, wherein:
L₁ is a direct bond, phenylene, biphenylene, or naphthylene.

5. The compound of claim 1, wherein:
L₂ is a direct bond, or phenylene.

6. The compound of claim 1, wherein:
Ar₁ and Ar₂ are each independently phenyl, biphenylyl, naphthyl, naphthyl phenyl, phenyl naphthyl, phenanthrenyl, carbazol-9-yl, 9-phenyl-9H-carbazolyl, dibenzofuranyl, or dibenzothiophenyl,
wherein the Ar₁ and Ar₂ are unsubstituted or substituted with at least one deuterium.

7. The compound of claim 1, wherein:
R₁ is phenyl, biphenylyl, terphenylyl, naphthyl, phenanthrenyl, dibenzofuranyl, or dibenzothiophenyl, and
wherein the R₁ is unsubstituted or substituted with at least one deuterium.

8. The compound of claim 1, wherein:
the compound of Chemical Formula 1 is any one compound selected from the group consisting of the following:

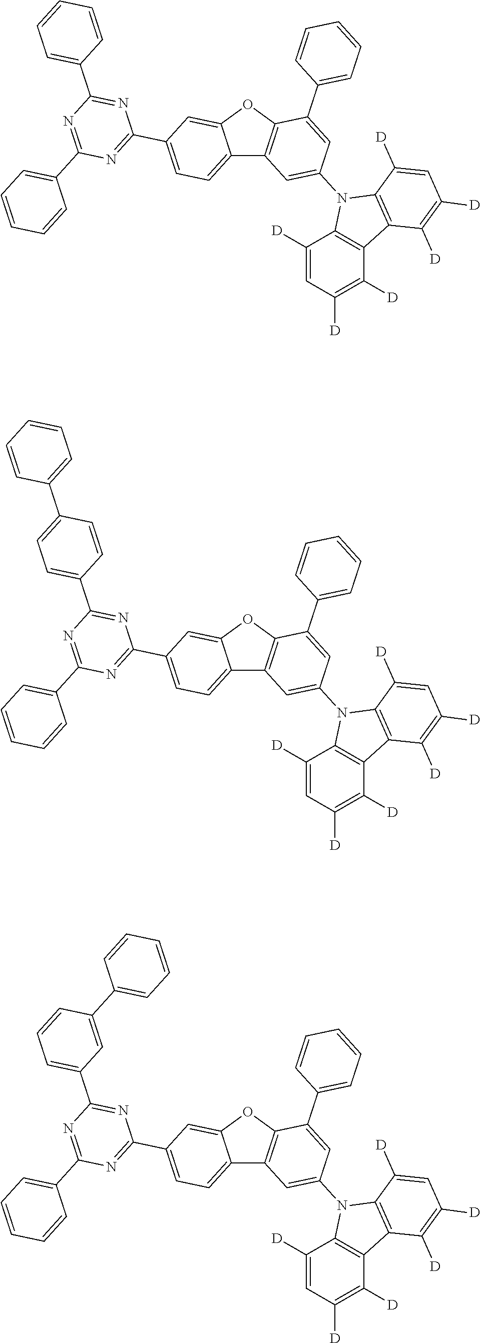 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6ccccc6)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc(-c6ccccc6)c5)n4)ccc3c2c1]

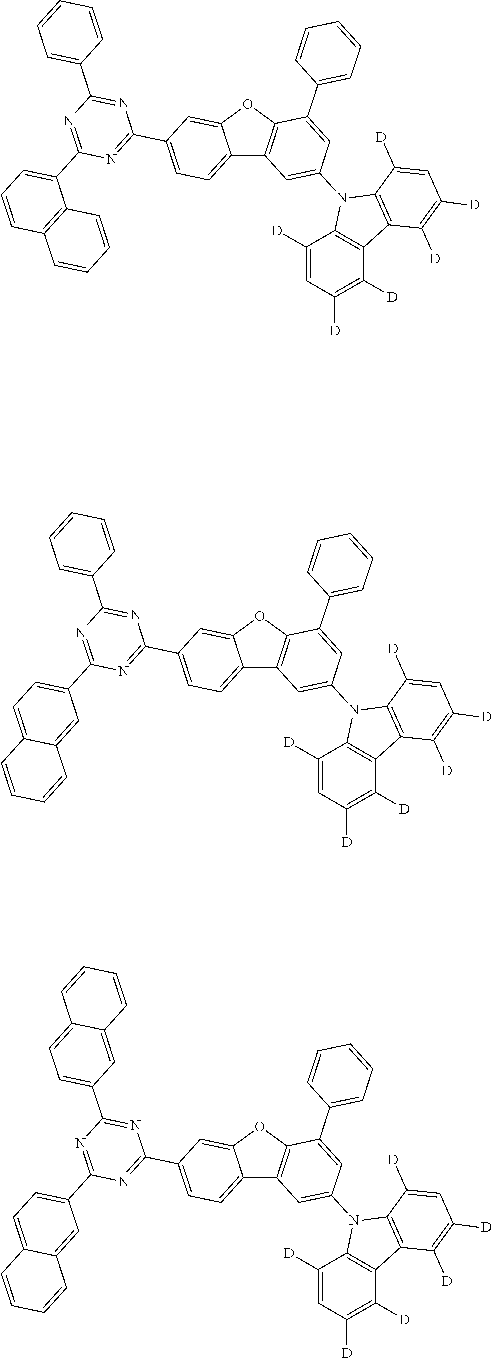 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6ccccc6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccc6ccccc6c5)nc(-c5ccc6ccccc6c5)n4)ccc3c2c1]

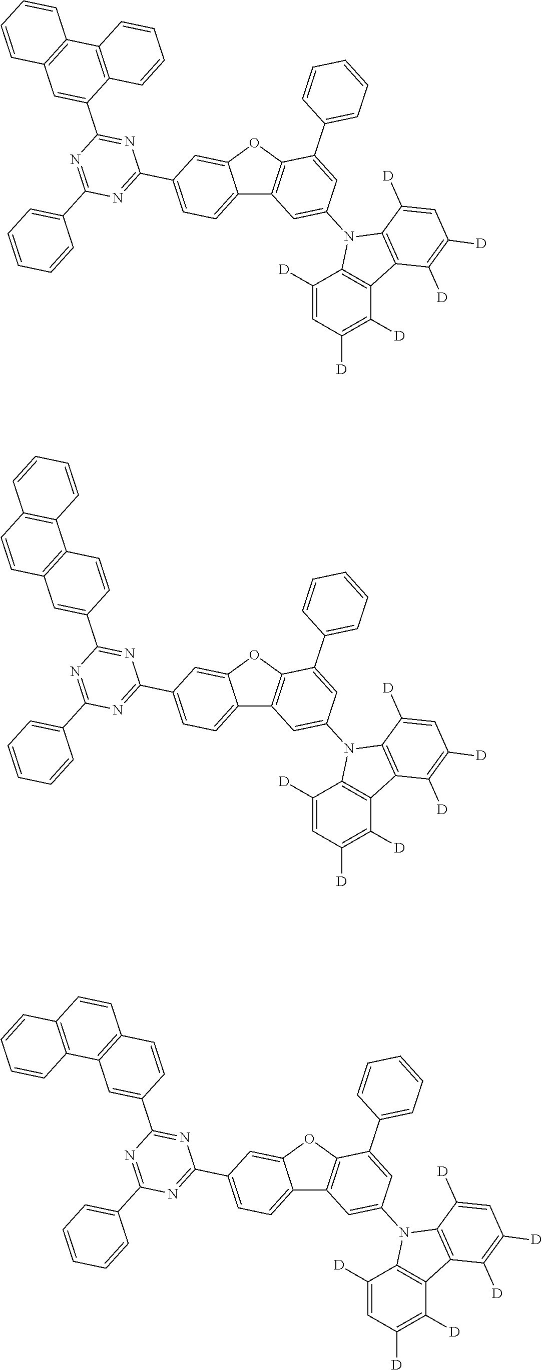 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5cc6ccccc6c6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(ccc7ccccc76)c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6ccc7ccccc7c6c5)n4)ccc3c2c1]

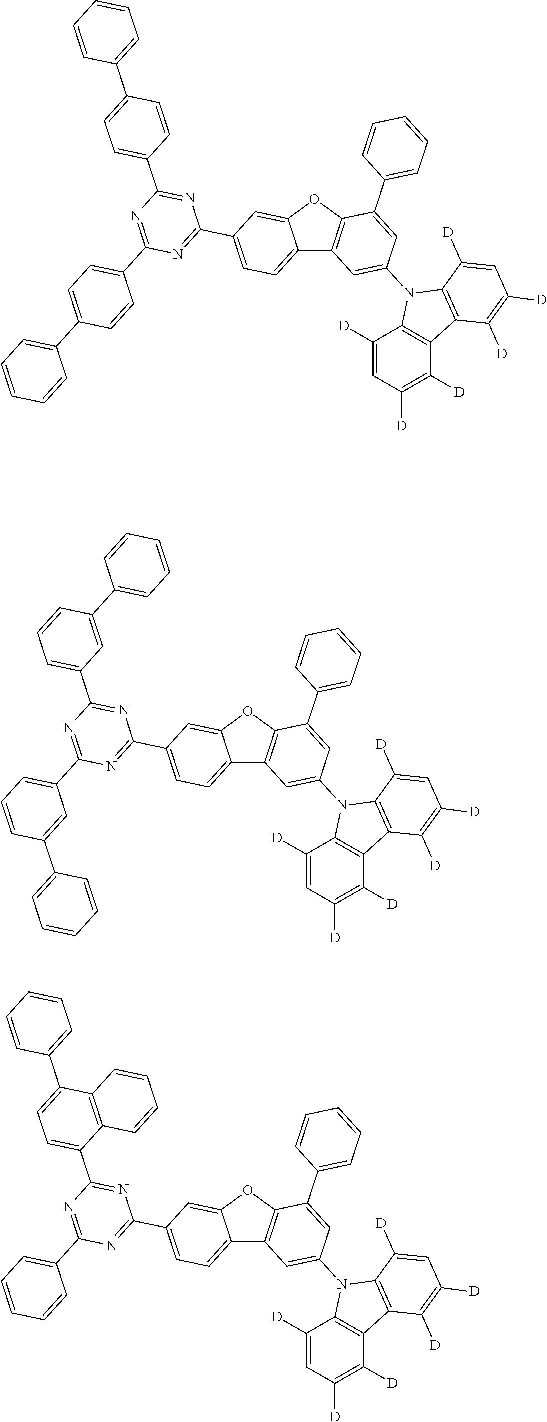 [SMILES: [2H]c1cc([2H])c2c(c1[2H])c1c([2H])c([2H])cc([2H])c1n2-c1cc(-c2ccccc2)c2oc3cc(-c4nc(-c5ccc(-c6ccccc6)cc5)nc(-c5ccc(-c6ccccc6)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5cccc(-c6ccccc6)c5)nc(-c5cccc(-c6ccccc6)c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6ccccc6)c6ccccc56)n4)ccc3c2c1]

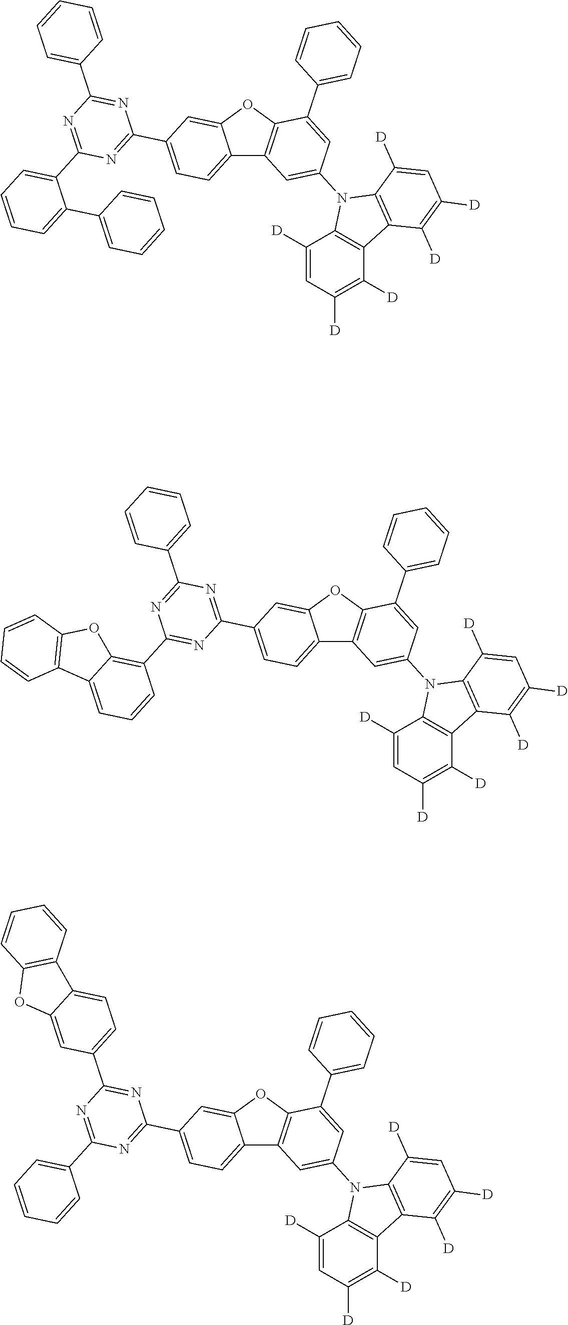 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccccc5-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5oc5ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)oc5ccccc56)n4)ccc3c2c1]

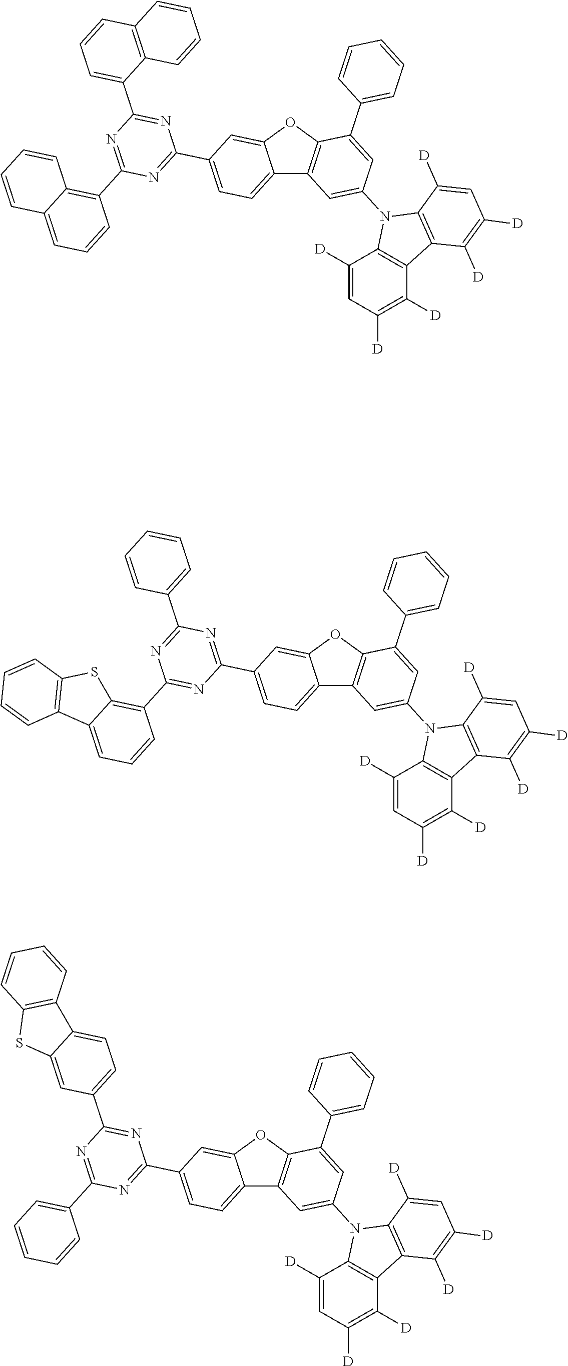 [SMILES: [2H]c1cc([2H])c2c(c1[2H])c1c([2H])c([2H])cc([2H])c1n2-c1cc(-c2ccccc2)c2oc3cc(-c4nc(-c5cccc6ccccc56)nc(-c5cccc6ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5sc5ccccc56)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)sc5ccccc56)n4)ccc3c2c1]

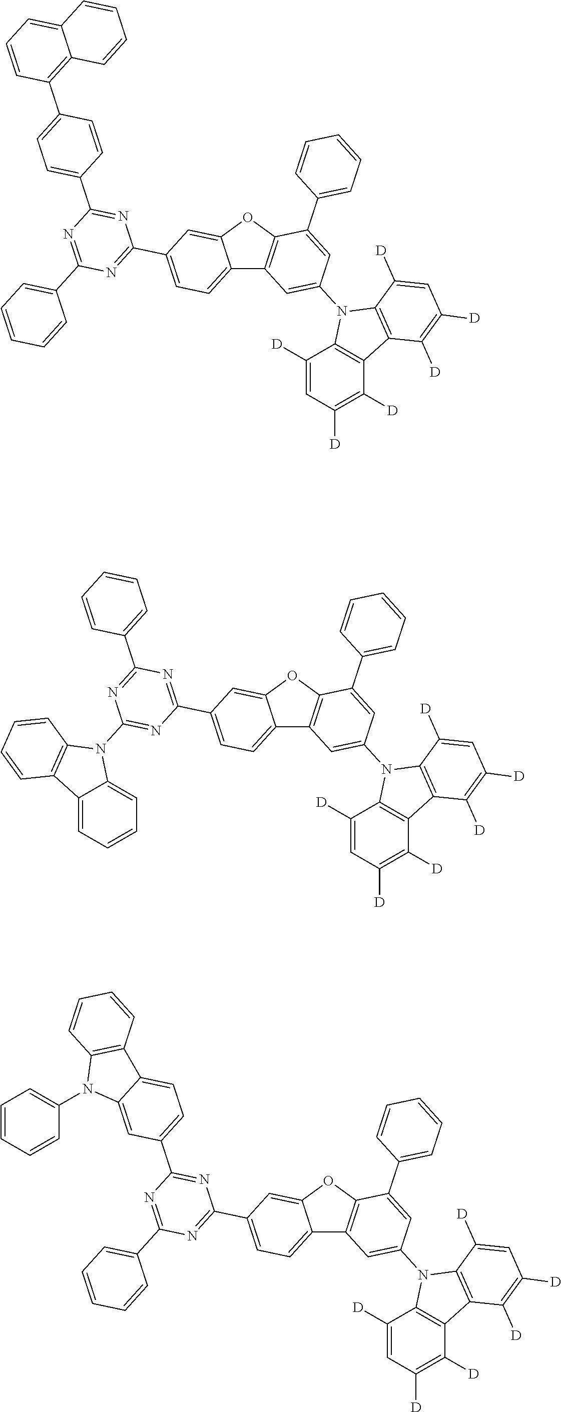 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc(-c6cccc7ccccc67)cc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-n5c6ccccc6c6ccccc65)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c7ccccc7n(-c7ccccc7)c6c5)n4)ccc3c2c1]

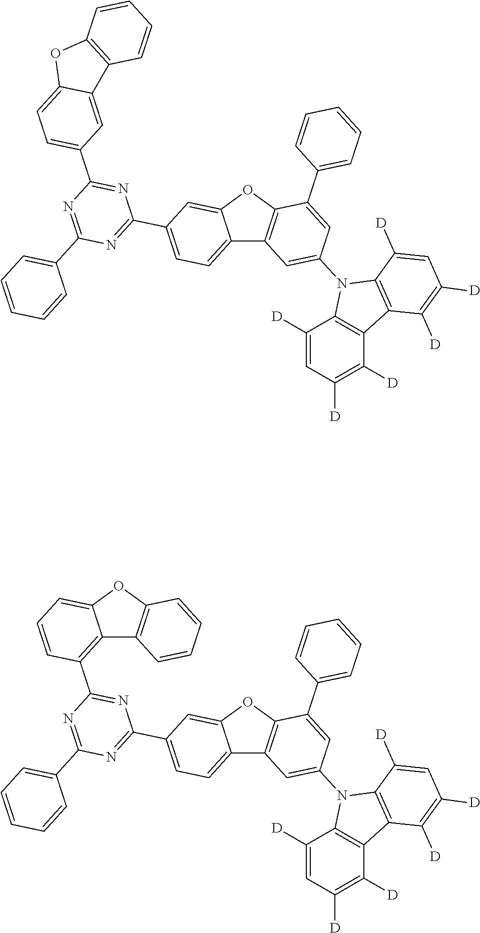 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6oc7ccccc7c6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6oc7ccccc7c56)n4)ccc3c2c1]

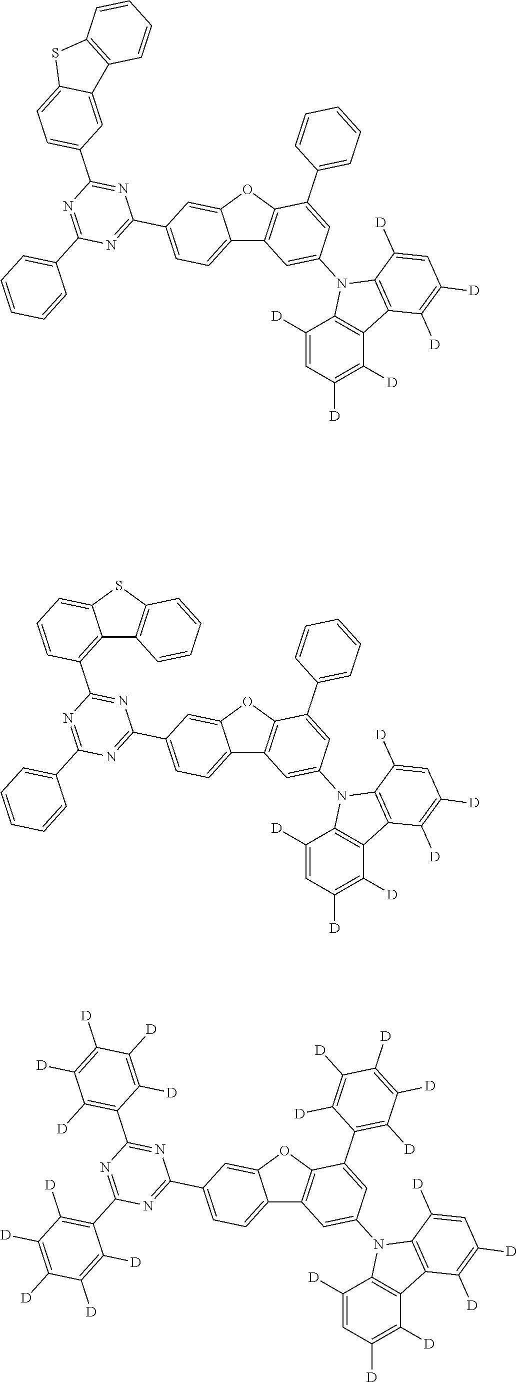 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6sc7ccccc7c6c5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6sc7ccccc7c56)n4)ccc3c2c1 ; [2H]c1cc([2H])c2c(c1[2H])c1c([2H])c([2H])cc([2H])c1n2-c1cc(-c2c([2H])c([2H])c([2H])c([2H])c2[2H])c2oc3cc(-c4nc(-c5c([2H])c([2H])c([2H])c([2H])c5[2H])nc(-c5c([2H])c([2H])c([2H])c([2H])c5[2H])n4)ccc3c2c1]

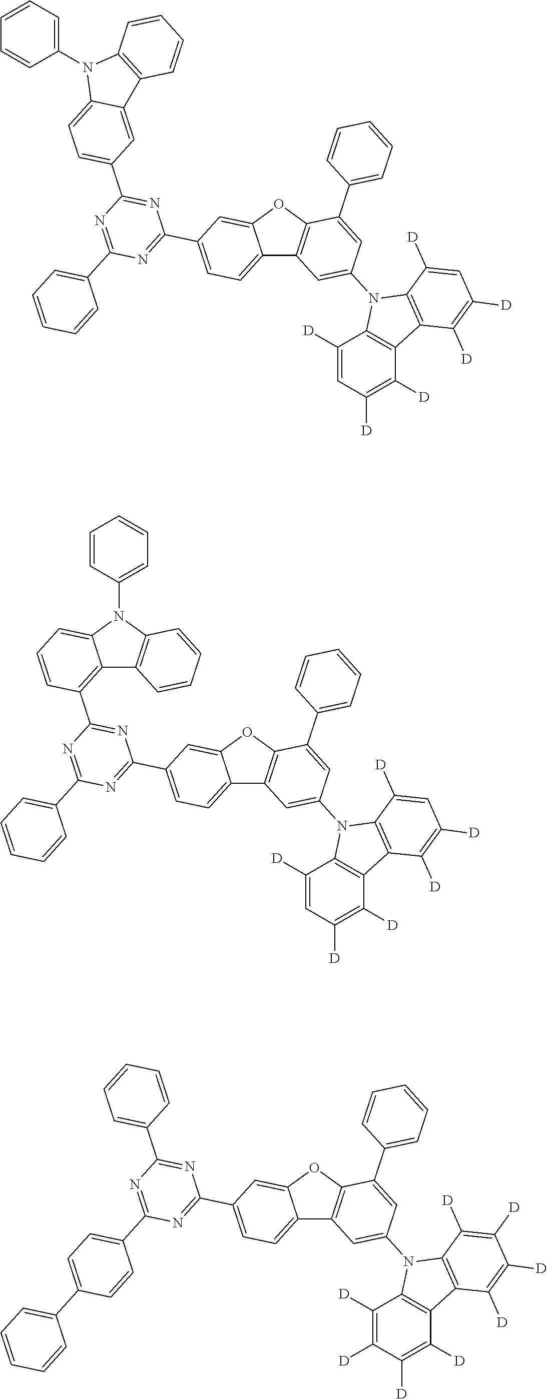 [SMILES: [2H]c1cc([2H])c2c(c1[2H])c1c([2H])c([2H])cc([2H])c1n2-c1cc(-c2ccccc2)c2oc3cc(-c4nc(-c5ccccc5)nc(-c5ccc6c(c5)c5ccccc5n6-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4nc(-c5ccccc5)nc(-c5cccc6c5c5ccccc5n6-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccc(-c6ccccc6)cc5)n4)ccc3c2c1]

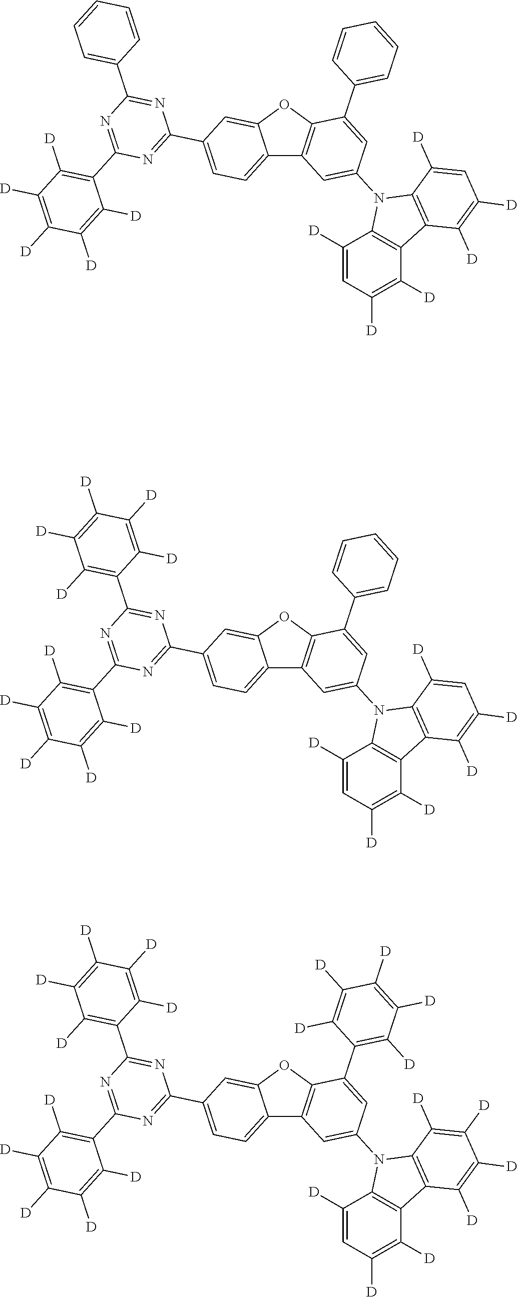

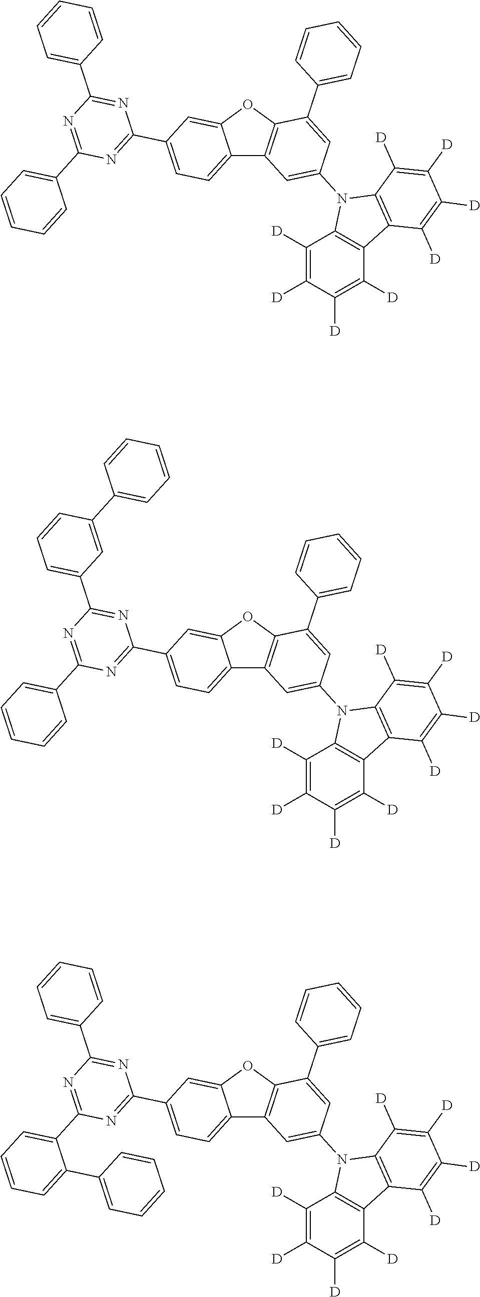 [SMILES: [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5cccc(-c6ccccc6)c5)n4)ccc3c2c1 ; [2H]c1c([2H])c([2H])c2c(c1[2H])c1c([2H])c([2H])c([2H])c([2H])c1n2-c1cc(-c2ccccc2)c2oc3cc(-c4nc(-c5ccccc5)nc(-c5ccccc5-c5ccccc5)n4)ccc3c2c1]

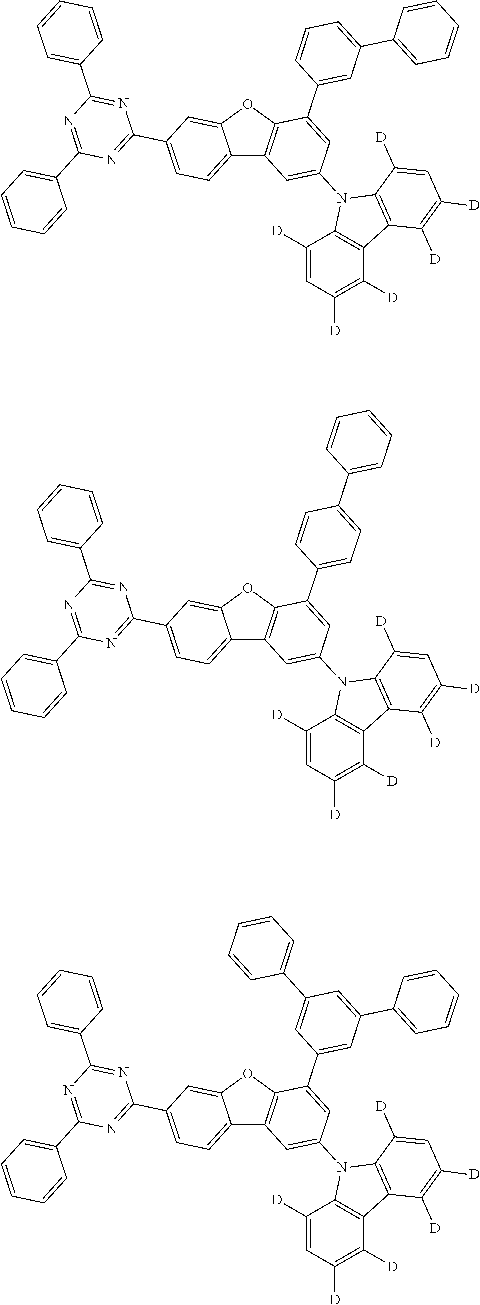 [SMILES: [2H]c1cc([2H])c2c(c1[2H])c1c([2H])c([2H])cc([2H])c1n2-c1cc(-c2cccc(-c3ccccc3)c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc(-c3ccccc3)cc2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cc(-c3ccccc3)cc(-c3ccccc3)c2)c2oc3cc(-c4nc(-c5ccccc5)nc(-c5ccccc5)n4)ccc3c2c1]

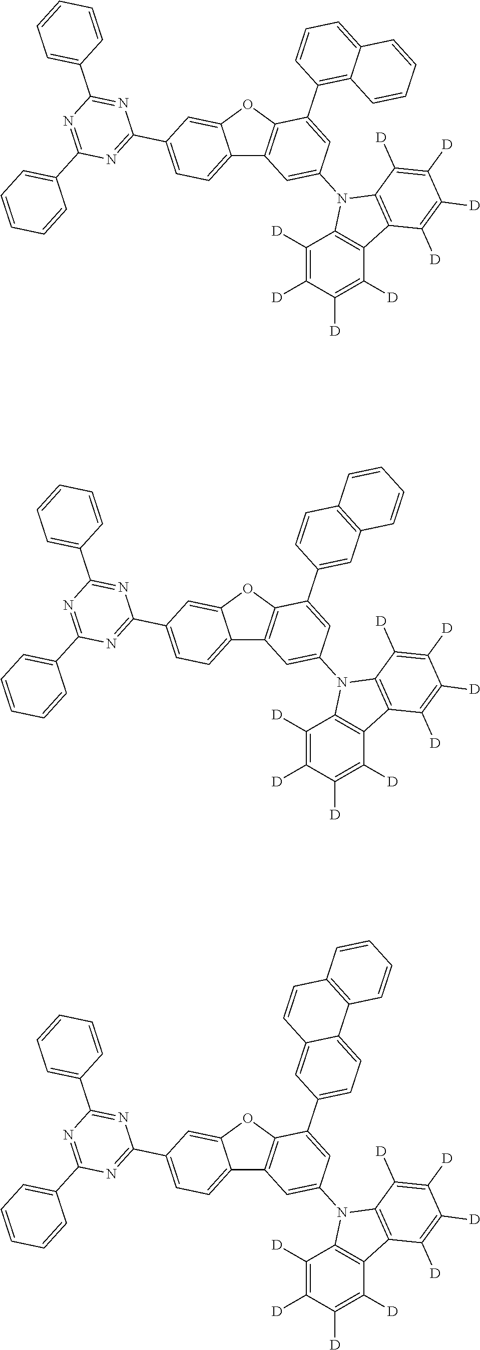 [SMILES: [2H]c1c([2H])c([2H])c2c(c1[2H])c1c([2H])c([2H])c([2H])c([2H])c1n2-c1cc(-c2cccc3ccccc23)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccc3ccccc3c2)c2oc3cc(-c4nc(-c5ccccc5)nc(-c5ccccc5)n4)ccc3c2c1 ; [2H]c1c([2H])c([2H])c2c(c1[2H])c1c([2H])c([2H])c([2H])c([2H])c1n2-c1cc(-c2ccc3c(ccc4ccccc43)c2)c2oc3cc(-c4nc(-c5ccccc5)nc(-c5ccccc5)n4)ccc3c2c1]

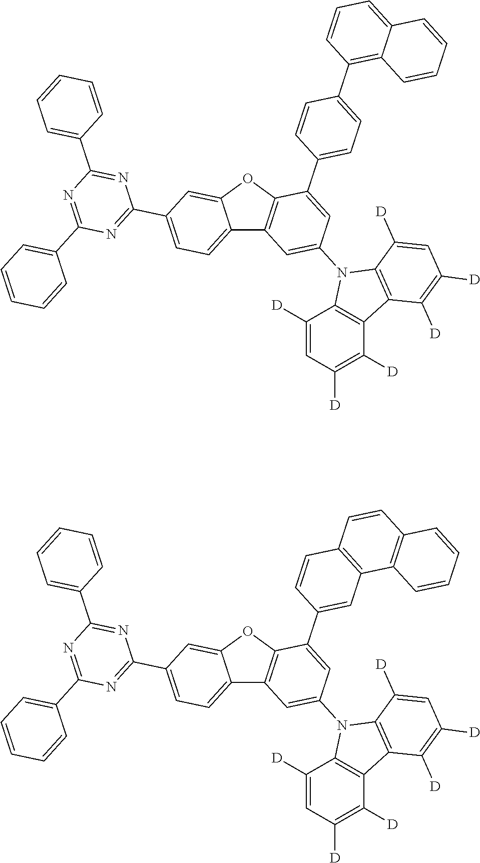 [SMILES: [2H]c1cc([2H])c2c(c1[2H])c1c([2H])c([2H])cc([2H])c1n2-c1cc(-c2ccc(-c3cccc4ccccc34)cc2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3ccc4ccccc4c3c2)c2oc3cc(-c4nc(-c5ccccc5)nc(-c5ccccc5)n4)ccc3c2c1]

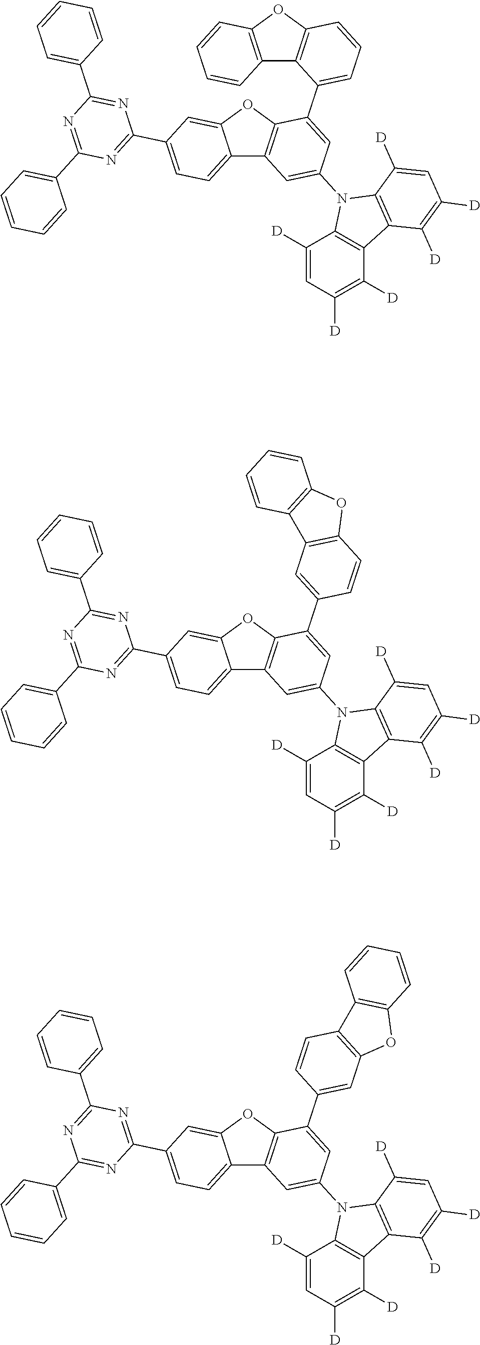 [SMILES: [2H]c1cc([2H])c2c(c1[2H])c1c([2H])c([2H])cc([2H])c1n2-c1cc(-c2cccc3oc4ccccc4c23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3oc4ccccc4c3c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3c(c2)oc2ccccc23)c2oc3cc(-c4nc(-c5ccccc5)nc(-c5ccccc5)n4)ccc3c2c1]

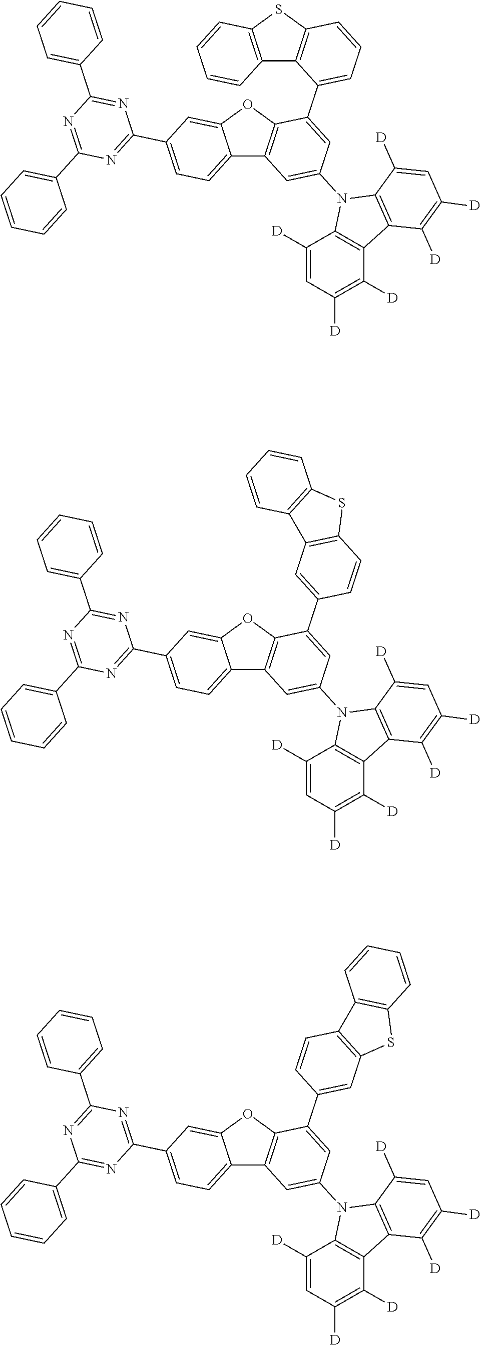 [SMILES: [2H]c1cc([2H])c2c(c1[2H])c1c([2H])c([2H])cc([2H])c1n2-c1cc(-c2cccc3sc4ccccc4c23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3sc4ccccc4c3c2)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccc3c(c2)sc2ccccc23)c2oc3cc(-c4nc(-c5ccccc5)nc(-c5ccccc5)n4)ccc3c2c1]

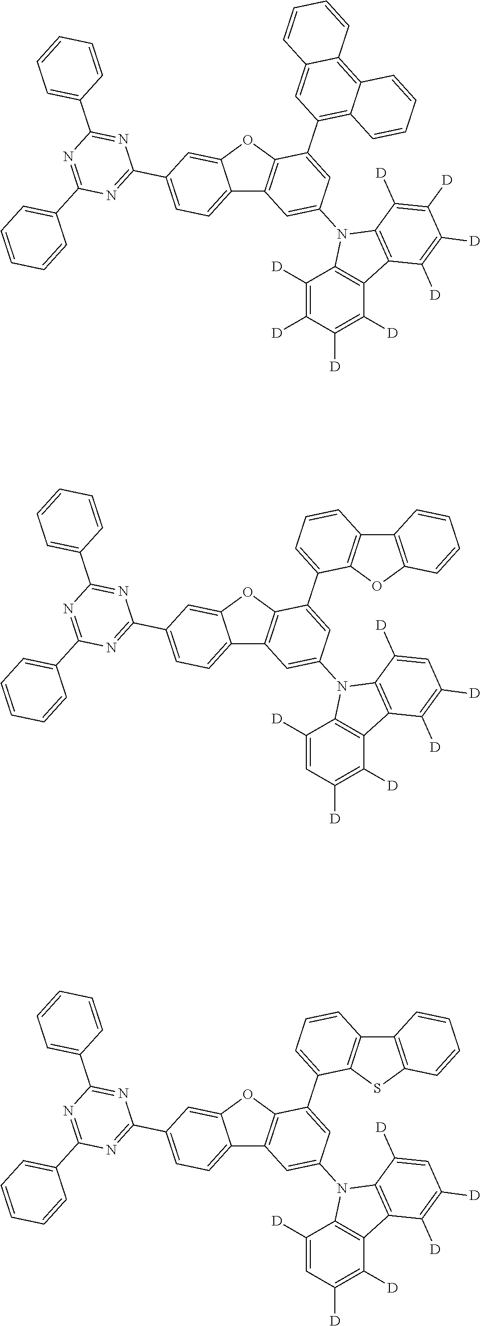 [SMILES: [2H]c1c([2H])c([2H])c2c(c1[2H])c1c([2H])c([2H])c([2H])c([2H])c1n2-c1cc(-c2cc3ccccc3c3ccccc23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cccc3c2oc2ccccc23)c2oc3cc(-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2cccc3c2sc2ccccc23)c2oc3cc(-c4nc(-c5ccccc5)nc(-c5ccccc5)n4)ccc3c2c1]

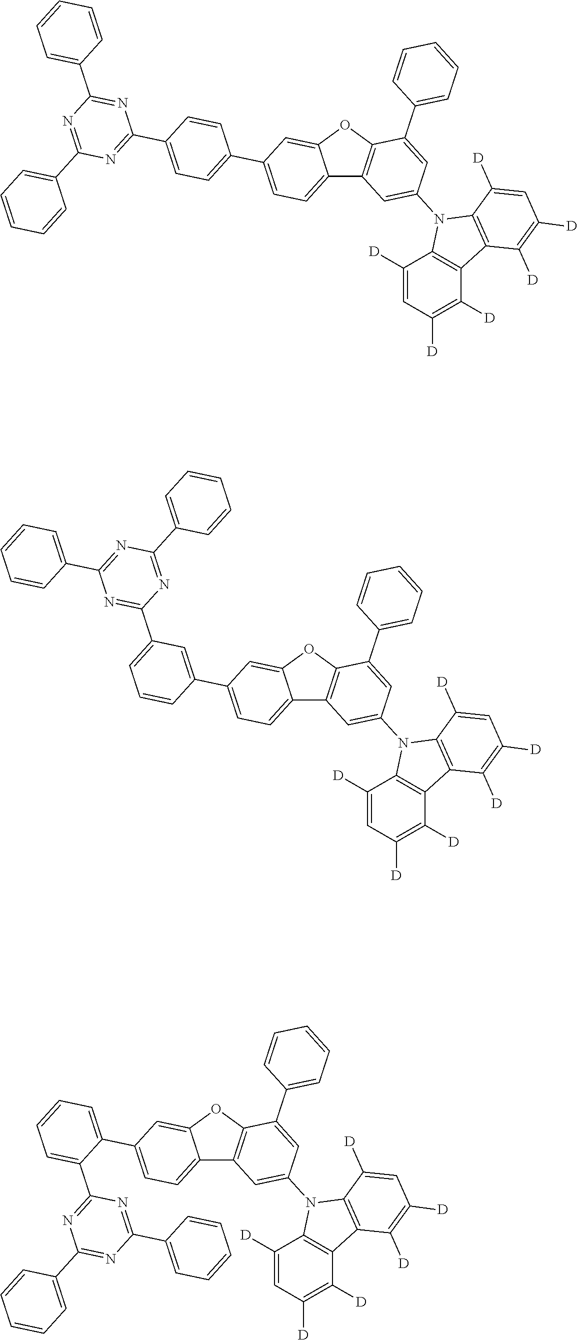 [SMILES: [2H]c1cc([2H])c2c(c1[2H])c1c([2H])c([2H])cc([2H])c1n2-c1cc(-c2ccccc2)c2oc3cc(-c4ccc(-c5nc(-c6ccccc6)nc(-c6ccccc6)n5)cc4)ccc3c2c1 ; [2H]c1cc([2H])c2c(c1[2H])c1c([2H])c([2H])cc([2H])c1n2-c1cc(-c2ccccc2)c2oc3cc(-c4cccc(-c5nc(-c6ccccc6)nc(-c6ccccc6)n5)c4)ccc3c2c1 ; [2H]c1cc([2H])c2c(c1[2H])c1c([2H])c([2H])cc([2H])c1n2-c1cc(-c2ccccc2)c2oc3cc(-c4ccccc4-c4nc(-c5ccccc5)nc(-c5ccccc5)n4)ccc3c2c1]

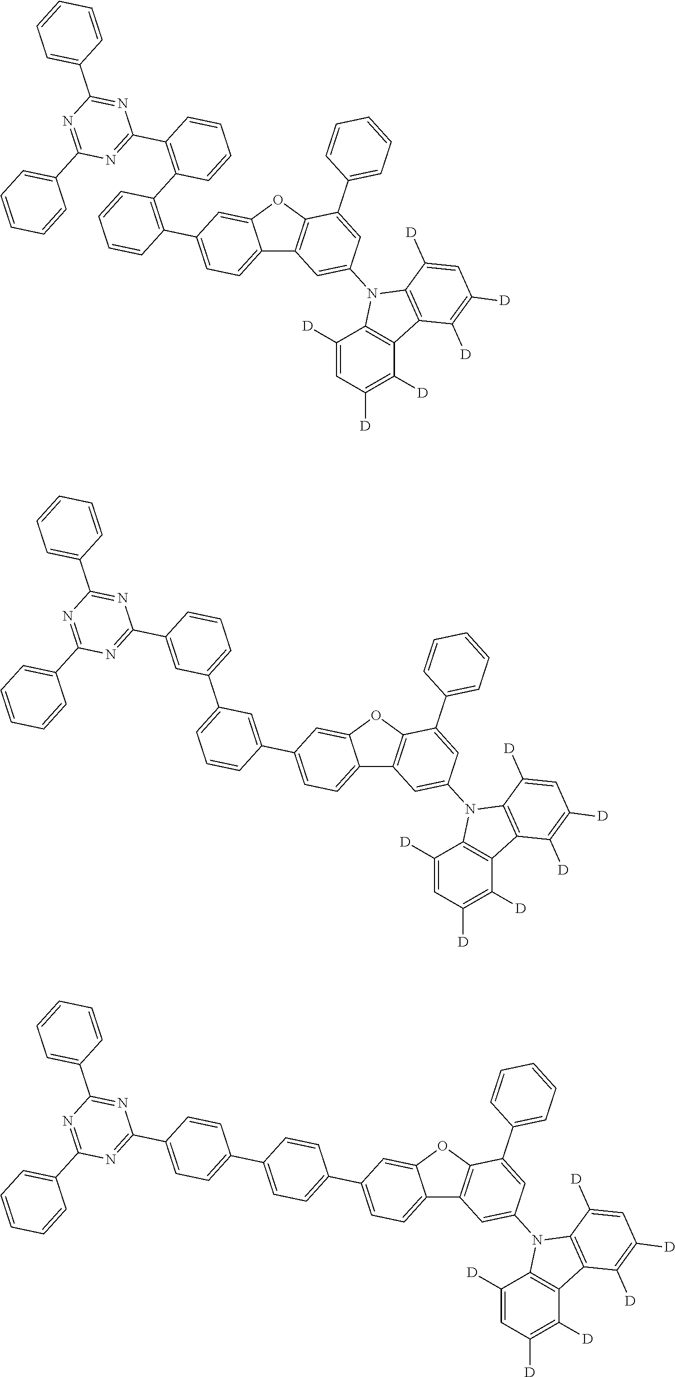 [SMILES: [2H]c1cc([2H])c2c(c1[2H])c1c([2H])c([2H])cc([2H])c1n2-c1cc(-c2ccccc2)c2oc3cc(-c4ccccc4-c4ccccc4-c4nc(-c5ccccc5)nc(-c5ccccc5)n4)ccc3c2c1 ; [2H]c1cc([2H])c2c(c1[2H])c1c([2H])c([2H])cc([2H])c1n2-c1cc(-c2ccccc2)c2oc3cc(-c4cccc(-c5cccc(-c6nc(-c7ccccc7)nc(-c7ccccc7)n6)c5)c4)ccc3c2c1 ; [2H]c1cc([2H])c2c(c1[2H])c1c([2H])c([2H])cc([2H])c1n2-c1cc(-c2ccccc2)c2oc3cc(-c4ccc(-c5ccc(-c6nc(-c7ccccc7)nc(-c7ccccc7)n6)cc5)cc4)ccc3c2c1]

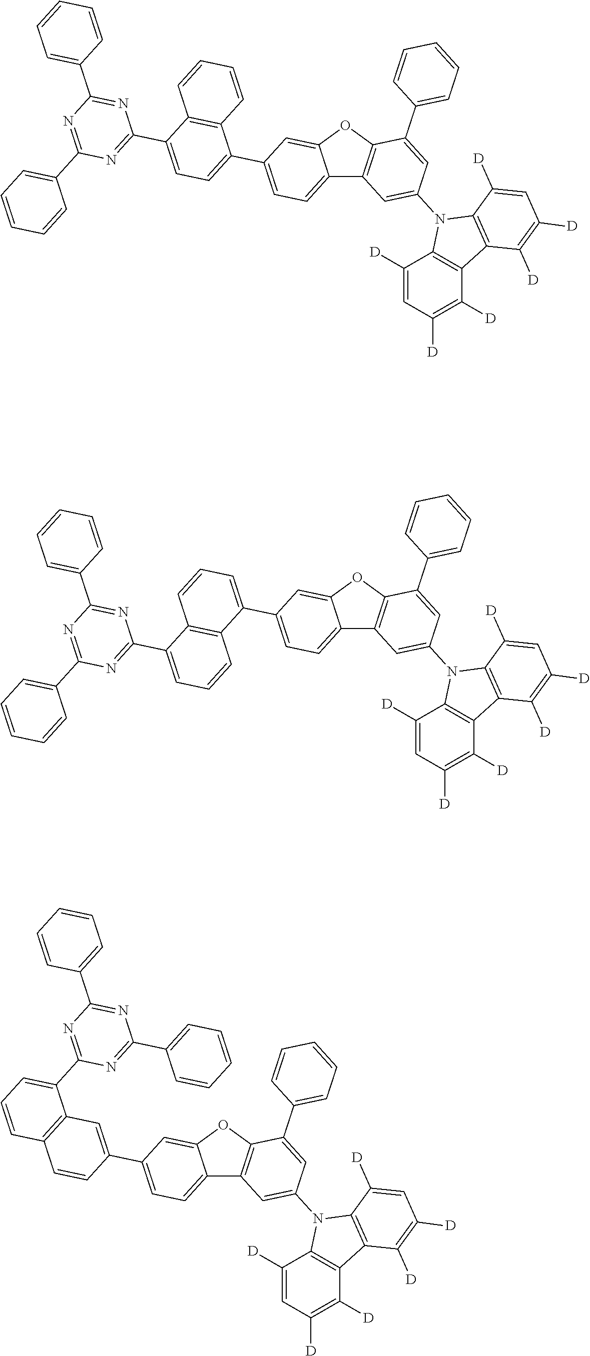 [SMILES: [2H]c1cc([2H])c2c(c1[2H])c1c([2H])c([2H])cc([2H])c1n2-c1cc(-c2ccccc2)c2oc3cc(-c4ccc(-c5nc(-c6ccccc6)nc(-c6ccccc6)n5)c5ccccc45)ccc3c2c1 ; [2H]c1cc([2H])c2c(c1[2H])c1c([2H])c([2H])cc([2H])c1n2-c1cc(-c2ccccc2)c2oc3cc(-c4cccc5c(-c6nc(-c7ccccc7)nc(-c7ccccc7)n6)cccc45)ccc3c2c1 ; [2H]c1cc([2H])c2c(c1[2H])c1c([2H])c([2H])cc([2H])c1n2-c1cc(-c2ccccc2)c2oc3cc(-c4ccc5cccc(-c6nc(-c7ccccc7)nc(-c7ccccc7)n6)c5c4)ccc3c2c1]

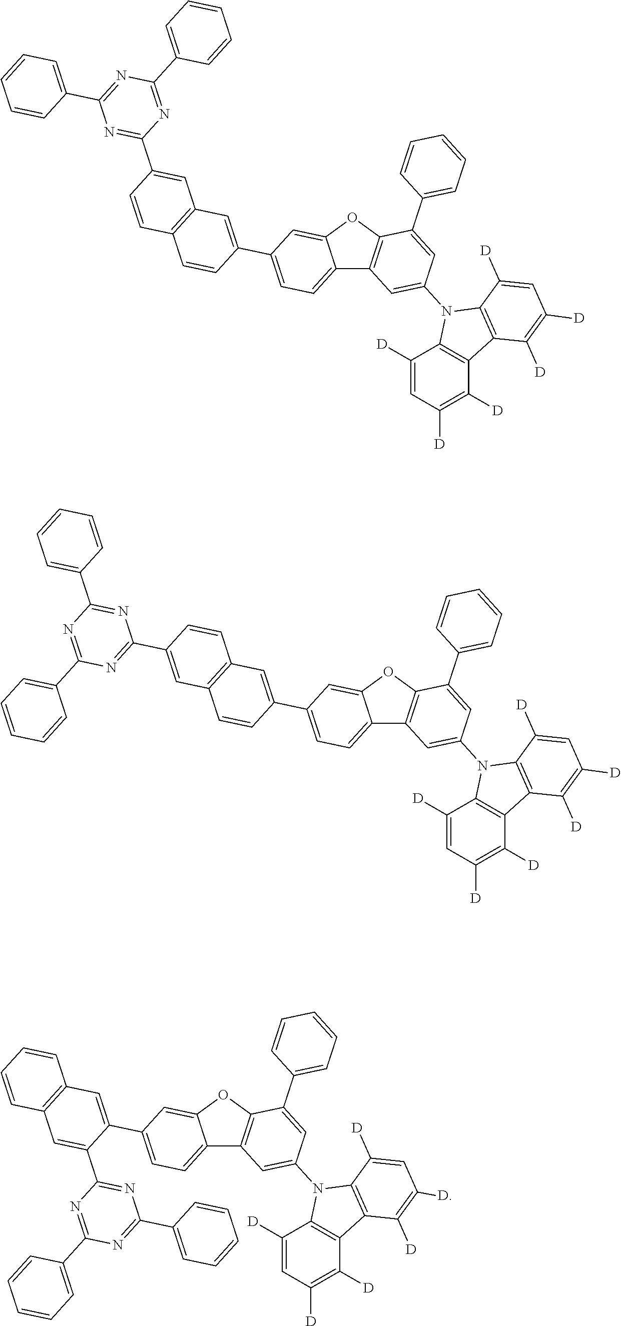

9. An organic light emitting device comprising: a first electrode; a second electrode that is provided opposite to the first electrode; and one or more organic material layers that are provided between the first electrode and the second electrode, wherein one or more layers of the organic material layers comprises the compound of claim 1.

10. The organic light emitting device of claim 9, wherein:
the organic material layer comprising the compound is a light emitting layer.
